SEQUENCE LISTING (21 sequences)

SEQ ID NO: 1 - DNA, 160 bp, Zea mays B73
atggacgacatcacccaacactttggagttggcgcaagcagccacggccatggccacggccagcaccaccatcatcaccaccaccaccacccgtgggcatcctcccctcagcgccgtcgtagcgccgctgccgccgctaccgccaagcgcaggcctgccg

SEQ ID NO: 2 - DNA, 160 bp, Zea mays Mo17
atggacgacatcacccaacactttggagttggcgcaagcagccacggccatggccacggcaagcaccacaatcatcaccaccaccaccacccgtgggcatcctcccctcagcgccgtcgtagcgccgctgccgccgctaccgccaagcgcaggcctgccg

SEQ ID NO: 3 - DNA, 160 bp, Sorghum bicolor
atggacgacatcacccaacactttggagttggcgcaagcagccacggccatggccacggccagcagacgacatcacccaaccaccaccacccgtgggcatcctcccctcagcgccgtcgtagcgccgctgccgccgccaccgccaagctcaggcctgccg

SEQ ID NO: 4 - DNA, 160 bp, Setaria italica
atggaggagatcacccaacactttggagttggcgcaagcagccacggccatggccacggccagcaccaccatcatcaccaccaccaccacccgtgggcatcctcccctcagcgccgtcgtagcgccgctgccgccgccaccgccaagctcaggcctgccg

SEQ ID NO: 5 - DNA, 160 bp, Andropogon gerardi
atggacgacatcacccaacactttggagttggcgcaagcagccacggccatggccacggccagcaccaccatcatcaccaccaccaccacccgtgggcatcctcccctcagcgccgtcgtagcgccgctgccgccgccaccgccaagctcaggcctgccg

SEQ ID NO: 6 - protein, 53 aa, Zea mays B73
MDDITQHFGVGASSHGHGHGQHHHHHHHHHPWASSPQRRRSAAAAATAKRRPA

SEQ ID NO: 7 - protein, 53 aa, Zea mays Mo17
MDDITQHFGVGASSHGHGHGKHHNHHHHHHPWASSPQRRRSAAAAATAKRRPA

SEQ ID NO: 8 - protein, 53 aa, Sorghum bicolor
MDDITQHFGVGASSHGHGHGQQTTSPNHHHPWASSPQRRRSAAAAATAKLRPA

SEQ ID NO: 9 - protein, 53 aa, Setaria italica
MEEITQHFGVGASSHGHGHGQHHHHHHHHHPWASSPQRRRSAAAAATAKLRPA

SEQ ID NO: 10 - protein, 53 aa, Andropogon gerardii
MDDITQHFGVGASSHGHGHGQHHHHHHHHHPWASSPQRRRSAAAAATAKLRPA

SEQ ID NO: 11 - DNA, 315 bp, Zea mays
atggacatcgggttccggatttgtgcttcgatcatatccagctgccagcttggtcgtcgacacgtttatttccacagttgtgctcccattcttgcttcgtcgattgtttcgttttggtgcacagctgatttcgtttgtgtcaaatgcgaacatccactgctcgaaccaccatggcgctcggctatagagcgtcatgacagcagctccccaagcgatgcaagccgcctgtttgacgaaatgtgtgagaagcaaaatagagacatcaatggtgttttcatgtgtctaatacattttaaaaaaaacatagctagatag

SEQ ID NO: 12 - DNA, 315 bp, Zea mays
atggacatctggttccggatttgtgcttcgatcatatccagctgccagtttggtcgtcgacacatttatgtccacagttgtgctcccattcttgcttcgtcgattgtttcgttttggtgcacagctgatttcgtttgtgtcaaatgcgaacattcactgctcgaaccaccatggcgctcggctacagggcgtcatgacagcagctccccaagcgatgcaagccgcatgtttgacgaaatgtgtgagaagcaaaatagagacatcaatggtgttttcatgtgtctaatacattttaaaaaaaacatagctagatag

SEQ ID NO: 13 - protein, 104 aa, Zea mays
MDIGFRICASIISSCQLGRRHVYFHSCAPILASSIVSFWCTADFVCVKCEHPLLEPPWRSAIERHDSSSPSDASRLFDEMCEKQNRDINGVFMCLIHFKKNIAR

SEQ ID NO: 14 - protein, 104 aa, Zea mays
MDIWFRICASIISSCQFGRRHIYVHSCAPILASSIVSFWCTADFVCVKCEHSLLEPPWRSATGRHDSSSPSDASRMFDEMCEKQNRDINGVFMCLIHFKKNIAR

SEQ ID NO: 15 - DNA, 366 bp, Zea mays
atgccttcttcctgcatccgtgggccatcgtcacgccatgaatgcgaaggtctcttcgccatcgagacctccgtcttcaagaaggaaaatgtctccctggctggccatgaggagaaactagccaggcacaggcagcacctcagccccctcctcagccagacggctactgtctcgaggcgtacagtggccgctcacctcgcggacgtgtctggctcgtcatcggcctccaatgccacgatcgcgctgctactccaatcgtcgctgacccgcatgatctccactgacttccgcaatgagtgcgggcaccttcggtcccatcagctgttaaggattacctcgcttatggtgttccacagagtgacatga

SEQ ID NO: 16 - DNA, 366 bp, Zea mays
atgccttcttcctgcctccgtgggccatcgtcacgccatgaatgcgaaggtctcttcgccatcgagacctccgtcttcaagaaggaaaaggtctccctggctggccatgaggagaaaccagccaggcacaagcagcacctcggccccctcttcagccagacggctactgtctcgaggcgtacagtggccgctcacctcgcgggcgtgtctggctcgtcatcggcctccaatgccacgatcgcgctgctactccaatcgtcgctgacctgcatgatctccactgacttccgcaatgagtgcgggcaccttcggtcccatccgctgttaaggattacctcgcttatggtgttccacagagtgacatga

SEQ ID NO: 17 - protein, 121 aa, Zea mays
MPSSCIRGPSSRHECEGLFAIETSVFKKENVSLAGHEEKLARHRQHLSPLLSQTATVSRRTVAAHLADVSGSSSASNATIALLLQSSLTRMISTDFRNECGHLRSHQLLRITSLMVFHRVT

SEQ ID NO: 18 - protein, 121 aa, Zea mays
MPSSCLRGPSSRHECEGLFAIETSVFKKEKVSLAGHEEKPARHKQHLGPLFSQTATVSRRTVAAHLAGVSGSSSASNATIALLLQSSLTCMISTDFRNECGHLRSHPLLRITSLMVFHRVT

SEQ ID NO: 19 - DNA, 201 bp, Zea mays
aagtctgtttgttttttttaatttcataaacttattaaaatgtcgcaggccaattttggaccctattgcttctgtattccacaaacttttctgtgggcgacctgcaagacctgaaggcacaggccagacattggatgggtcacagttccctggttcaggctccactgaggcaaacaggaggaggtgcggattccctttttc

SEQ ID NO: 20 - DNA, 201 bp, Zea mays
ggcgcagcctacttccgatgctgtcgtcgacgagggaagcggcgggaagagcatcgtcgcgtccccctggagctgccactcgtccgcggcggccgtggacagcgtgtccgcggcgtttccagggatggctccgccggaccggacgatgtccgtgaggtctccgccgccggcctggtcgccctccatcctcggaaggaagta

SEQ ID NO: 21 - DNA, 201 bp, Zea mays
aacaatctacttgcagtgcaatatttctaaattgtacttgcagtgcaagatttcaaatcaatctacttaccgtgccagatttcaggggcttagacaggatatcattgcagaccttgtggatcaatgccgttcttacaagcaaagagtagtgcagcttgtcaacagtacctcgtaagttaccttgatgactcttttctagtt

(19) United States
(12) Patent Application Publication
(10) Pub. No.: US 2022/0277807 A1
(43) Pub. Date: Sep. 1, 2022

(54) METHODS AND SYSTEMS FOR ASSESSING GENETIC VARIANTS

(71) Applicant: Inari Agriculture Technology, Inc., Cambridge, MA (US)

(72) Inventors: Karl Anton Grothe KREMLING, Cambridge, MA (US); Stephen GOFF, Wake Forest, NC (US); Wenbin MEI, Belmont, MA (US); Ruijuan LI, Somerville, MA (US); Ross Everett ALTMAN, Boston, MA (US); Zachary Hartford LEMMON, Newtonville, MA (US)

(73) Assignee: Inari Agriculture Technology, Inc., Cambridge, MA (US)

(21) Appl. No.: 17/636,332

(22) PCT Filed: Aug. 21, 2020
(86) PCT No.: PCT/US2020/047464

§ 371 Date: Feb. 17, 2022

Related U.S. Application Data
(60) Provisional application No. 62/988,252, filed on Mar. 11, 2020.
(60) Provisional application No. 62/890,352, filed on Aug. 22, 2019.

(51) Int. Cl. G16B 20/50; G16B 40/00

(52) U.S. Cl. CPC G16B 20/50; G16B 40/00

(57) ABSTRACT

Provided herein are methods for assessing genetic variants for use in genetically improving organisms and in human genetics and medicine. Also provided herein are systems for implementing such methods, as well as computer-readable storage media storing instructions for performing such methods.

### CROSS-REFERENCE TO RELATED APPLICATIONS

This application claims priority to U.S. Provisional Patent Application No. 62/890,352, filed Aug. 22, 2019, and U.S. Provisional Patent Application No. 62/988,252, filed Mar. 11, 2020, the entireties of which are incorporated herein by reference.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name 165362000140SEQLIST.txt, date recorded: Aug. 18, 2020, size: 12 KB).

### FIELD

The present disclosure relates generally to genetics, and more specifically to methods and systems of assessing genetic variants for use in genetic improvement of organisms and human genetics and medicine.

### BACKGROUND

The traditional phenotype-based breeding and the more recent genomic selection technique have made significant achievement in improving economically valuable and genetically complex traits (e.g. highly polygenic or controlled by more than 50 genomic loci) in agricultural species, for example, yield performance in maize (Heffner et al., Crop Science, 2009; 49(1):1-12). However, further progress in genetic improvement of such complex traits requires a better understanding of the underlying genetic variants and functions thereof.

Various efforts have been attempted to address this issue. The use of computational techniques and machine learning methods has aided prediction of the phenotypic effects of genetic variants. On the other hand, advances in biotechnology, such as genome editing, have facilitated testing of the phenotypic effects of genetic variants. However, current methods and systems are limited in efficiency and accuracy of assessing genetic variants for effective use in genetically improving agricultural species, as well as in human genetics and medicine.

Accordingly, there is a need for improved methods and systems for assessing genetic variants. The assessed genetic variants can then be prioritized and used as candidates for genetic modification or targets for selection to improve desirable traits (e.g. yield performance) in the agricultural species, as well as for use in human genetics and medicine (e.g. as a target in precision medicine).

### BRIEF SUMMARY

Provided herein are methods for assessing genetic variants for use in genetically improving organisms. Also provided herein are systems for implementing such methods, as well as computer-readable storage media storing instructions for performing such methods.

In one aspect, provided herein is a method for improving performance of an organism, including: a) providing a plurality of genetic variants in the genome of the organism; b) predicting the effects of the genetic variants on the performance of the organism using a statistical model; c) altering one or more of the genetic variants in the genome of the organism; d) identifying an impact of the alteration on an endophenotype, wherein the endophenotype is a quantifiable phenotype at the sub-organismal level that can be measured by a biochemical, gene expression, or protein level assay, or visually via microscopy; e) updating the statistical model using the identified endophenotypic impact; f) optionally repeating steps c) to e) for one or more times; g) determining the genetic variants having a predicted negative effect on the performance of the organism using the updated statistical model; and h) modifying in the genome one or more of the genetic variants having a predicted negative effect on the performance of the organism, thereby improving performance of an organism.

In another aspect, provided herein is a method for selecting an organism with improved performance in a population, including: a) providing a population of organisms; b) providing a plurality of genetic variants of the population; c) predicting the effects of the genetic variants on the performance of the organisms using a statistical model; d) altering one or more of the genetic variants in one or more of the organisms; e) identifying an impact of the alteration on an endophenotype, wherein the endophenotype is a quantifiable phenotype at the sub-organismal level that can be measured by a biochemical, gene expression, or protein level assay, or visually via microscopy; f) updating the statistical model using the identified endophenotypic impact; g) optionally repeating steps d) to f) for one or more times; h) determining the genetic variants having predicted positive effects on the performance of the organisms using the updated statistical model; and i) selecting in the population an organism comprising one or more of the genetic variants having predicted positive effects on the performance of the organisms, selecting an organism with improved performance in a population.

In yet another aspect, provided herein is a method for removing an underperforming organism from a population, including: a) providing a population of organisms; b) providing a plurality of genetic variants of the population; c) predicting the effects of the genetic variants on the performance of the organisms using a statistical model; d) altering one or more of the genetic variants in one or more of the organisms; e) identifying an impact of the alteration on an endophenotype, wherein the endophenotype is a quantifiable phenotype at the sub-organismal level that can be measured by a biochemical, gene expression, or protein level assay, or visually via microscopy; f) updating the statistical model using the identified endophenotypic impact; g) optionally repeating steps d) to f) for one or more times; h) determining the genetic variants having predicted negative effects using the updated statistical model; and i) removing from the population an organism comprising one or more of the genetic variants having predicted negative effects on the performance of the organisms, thereby removing an underperforming organism from a population.

In still another aspect, provided herein is a method for prioritizing genetic variants based on predicted effects on performance of an organism, including: a) providing a plurality of genetic variants in the genome of the organism; b) predicting the effects of the genetic variants on the performance of the organism using a statistical model; c) altering one or more of the genetic variants in the genome of the organism; d) identifying an impact of the alteration on an endophenotype, wherein the endophenotype is a quantifiable phenotype at the sub-organismal level that can be measured by a biochemical, gene expression, or protein level assay, or visually via microscopy; e) updating the statistical model using the identified endophenotypic impact; f) optionally repeating steps c) to e) for one or more times; and g) prioritizing the genetic variants based on the magnitudes of the predicted effects on the performance of organism using the updated statistical model.

In some embodiments, the organism is maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, or a biomass crop. In some embodiments that may be combined with the foregoing, the performance is yield, overall fitness, biomass, photosynthetic efficiency, nutrient use efficiency, heat tolerance, drought tolerance, herbicide tolerance, disease resistance.

In some embodiments, the organism is cattle, sheep, goat, horse, pig, chicken, duck, goose, rabbit, or fish. In some embodiments that may be combined with the foregoing, the growth rate, feed use efficiency, meat yield, meat quality, milk yield, milk quality, egg yield, egg quality, wool yield, or wool quality.

In some embodiments that may be combined with any of the preceding embodiments, the performance is a quantitative trait.

In some embodiments that may be combined with any of the preceding embodiments, the genetic variants are identified by a linkage study. In some embodiments that may be combined with any of the preceding embodiments, the genetic variants are identified by an association study. In some embodiments, the association study is a genome-wide association study (GWAS) or a transcriptome-wide association study (TWAS).

In some embodiments that may be combined with any of the preceding embodiments, the statistical model is a linear regression model, a logistic regression model, a ridge regression model, a lasso regression model, an elastic net regression model, a decision tree model, a gradient boosted tree model, a neural network model, or a support vector machine (SVM) model. In some embodiments that may be combined with any of the preceding embodiments, the statistical model comprises a feature based on evolutionary conservation of the genetic variants. In some embodiments, the evolutionary conservation is determined by sequence alignment in a genic or an intergenic region. In some embodiments that may be combined with any of the preceding embodiments, the statistical model comprises a feature based on functional impact of amino acid change of the genetic variants. In some embodiments, the functional impact of amino acid change is weighted according to the blocks substitution matrix (BLOSUM). In some embodiments that may be combined with any of the preceding embodiments, the statistical model comprises a feature based on functional impact of protein conformation and/or stability of the genetic variants. In some embodiments, the functional impact of protein conformation and/or stability is determined by a Monte Carlo search for minimal free energy. In some embodiments, the functional impact of protein conformation and/or stability is predicted by learning a representation of amino acid order from existing proteins in higher dimensional space. In some embodiments that may be combined with any of the preceding embodiments, the statistical model comprises a feature based on adjacency to a selective sweep region of the genetic variants. In some embodiments, the selective sweep region is determined by a decrease of pairwise nucleotide diversity π or linkage disequilibrium relative to the rest of the genome. In some embodiments that may be combined with any of the preceding embodiments, the statistical model comprises a feature based on outlier status of an endophenotype associated with a genetic variant that is physically proximal or proximal within a gene network. In some embodiments that may be combined with any of the preceding embodiments, the feature is a numeric or categorical value associated with a specific allele at a genomic locus.

In some embodiments that may be combined with any of the preceding embodiments, the alteration is achieved by genome editing. In some embodiments, the genome editing is achieved by a clustered regularly interspersed short palindromic repeats (CRISPR) system, a transcription activator-like effector nuclease (TALEN) system, or a zinc finger nuclease (ZFN) system.

In some embodiments that may be combined with any of the preceding embodiments, the alteration is achieved by creation of novel haplotype combinations from genetic recombination during meiosis.

In some embodiments that may be combined with any of the preceding embodiments, the endophenotype is messenger RNA (mRNA) abundance. In some variations, the endophenotype is gene transcript splicing ratio. In some variations, the endophenotype is protein abundance. In some variations, the endophenotype is micro RNA (miRNA) or small RNA (siRNA) abundance. In some variations, the endophenotype is translational efficiency. In some variations, the endophenotype is ribosome occupancy. In some variations, the endophenotype is protein modification. In some variations, the endophenotype is metabolite abundance. In some variations, the endophenotype is allele specific expression (ASE).

In certain aspects, the present invention provides an organism with improved performance produced or selected by any one of the preceding methods.

In yet some other aspects, provided herein is a computer-implemented method for assessing genetic variants for use in genetic improvement of an organism, including: a) receiving a dataset comprising a plurality of genetic variants of the organism; and b) performing a prediction of the effects of the genetic variants using a statistical model comprising one or more initial rules that associate the genetic variants with performance of the organism. In some embodiments, the method further includes updating the statistical model with one or more new rules, wherein the one or more new rules are based on data generated from an endophenotype, wherein the endophenotype is a quantifiable phenotype at the sub-organismal level that can be measured by a biochemical, gene expression, or protein level assay, or visually via microscopy. In some embodiments, the statistical model is a linear regression model, a logistic regression model, a ridge regression model, a lasso regression model, an elastic net regression model, a decision tree model, a gradient boosted tree model, a neural network model, or a support vector machine (SVM) model. In some embodiments, the one or more initial rules or the one or more new rules comprise evolutionary conservation, functional impact of amino acid change, functional impact of protein conformation and/or stability, adjacency to selective sweep regions, outlier status of an endophenotype associated with a genetic variant that is physically proximal or proximal within a gene network, or a combination thereof. In some embodiments, the endophenotype is messenger RNA (mRNA) abundance, gene transcript splicing ratio, protein abundance, micro RNA (miRNA) or small RNA (siRNA) abundance, translational efficiency, ribosome occupancy, protein modification, metabolite abundance, allele specific expression (ASE), or a combination thereof.

In yet some other aspects, provided herein is a computer-readable storage medium storing computer-executable instructions, including: a) instructions for applying a statistical model to a dataset, wherein the dataset comprises a plurality of genetic variants of an organism, and wherein the statistical model comprises one or more initial rules that associate the genetic variants with performance of the organism; and b) instructions for calculating an effect value related to the performance of the organism for each of the genetic variants. In some embodiments, the computer-readable storage medium further includes instructions for updating the statistical model with at least one new rule, wherein at least one new rule is based on data generated from an endophenotype, wherein the endophenotype is a quantifiable phenotype at the sub-organismal level that can be measured by a biochemical, gene expression, or protein level assay, or visually via microscopy. In some embodiments, the statistical model is a linear regression model, a logistic regression model, a ridge regression model, a lasso regression model, an elastic net regression model, a decision tree model, a gradient boosted tree model, a neural network model, or a support vector machine (SVM) model. In some embodiments, the one or more initial rules or the one or more new rules comprise evolutionary conservation, functional impact of amino acid change, functional impact of protein conformation and/or stability, adjacency to selective sweep regions, endophenotype outlier status of the genetic variants, or a combination thereof. In some embodiments, the endophenotype is messenger RNA (mRNA) abundance, gene transcript splicing ratio, protein abundance, micro RNA (miRNA) or small RNA (siRNA) abundance, translational efficiency, ribosome occupancy, protein modification, metabolite abundance, allele specific expression (ASE), or a combination thereof.

In yet some other aspects, provided herein is a system for assessing genetic variants for use in genetic improvement of an organism, including: a) a computer-readable storage medium storing a database comprising a plurality of genetic variants of the organism; b) a computer-readable storage medium storing computer-executable instructions, including: i) instructions for applying a statistical model to the dataset, wherein the statistical model comprises one or more initial rules that associate the genetic variants with performance of the organism; and ii) instructions for calculating an effect value related to the performance of the organism for each of the genetic variants; and c) a processor configured to execute the computer-executable instructions stored in the computer-readable storage medium. In some embodiments, the computer-readable storage medium further includes instructions for updating the statistical model with one or more new rules, wherein the one or more new rules are based on data generated from an endophenotype, wherein the endophenotype is a quantifiable phenotype at the sub-organismal level that can be measured by a biochemical, gene expression, or protein level assay, or visually via microscopy. In some embodiments, the statistical model is a linear regression model, a logistic regression model, a ridge regression model, a lasso regression model, an elastic net regression model, a decision tree model, a gradient boosted tree model, a neural network model, or a support vector machine (SVM) model. In some embodiments, the one or more initial rules or the one or more new rules comprise evolutionary conservation, functional impact of amino acid change, functional impact of protein conformation and/or stability, adjacency to selective sweep regions, outlier status of an endophenotype associated with a genetic variant that is physically proximal or proximal within a gene network, or a combination thereof. In some embodiments, the endophenotype is messenger RNA (mRNA) abundance, gene transcript splicing ratio, protein abundance, micro RNA (miRNA) or small RNA (siRNA) abundance, translational efficiency, ribosome occupancy, protein modification, metabolite abundance, allele specific expression (ASE), or a combination thereof.

In yet some other aspects, provided herein is a method for prioritizing genetic variants, comprising: a) providing a plurality of genetic variants in the genome of an organism; b) predicting the effects of the genetic variants on the performance of the organism using an endophenotype; and c) prioritizing the genetic variants based on the magnitudes of the predicted effects on the performance of the organism. In some embodiments, the method further comprises altering one or more of the prioritized genetic variants in the organism. In some embodiments, the method further comprises selecting one or more of the prioritized genetic variants from a population of the organisms. In some embodiments, the endophenotype is allele specific expression (ASE). In some embodiments, the statistical model comprises calculating the effect of a genetic variant on the biological function of a protein. In some embodiments, the calculated effect of a genetic variant is a likelihood ratio test P-value, a Protein Variation Effect Analyzer (PROVEAN) score, or a Sorting Intolerant from Tolerant (SIFT) score. In some embodiments, the organism is maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, or a biomass crop. In some embodiments, the organism is hybrid maize. In some embodiments, the performance of the organism is yield, overall fitness, biomass, photosynthetic efficiency, nutrient use efficiency, heat tolerance, drought tolerance, herbicide tolerance, or disease resistance. In some embodiments, the genetic variants comprise a deleterious allele that confers or correlates with a negative effect to the performance of the organism. In some embodiments, the deleterious allele is overexpressed or underexpressed in the organism in comparison to a control organism. In some embodiments, the control organism is an inbred line. In some embodiments, the genetic variants are homozygous or heterozygous in the organism. In some embodiments, the genetic variants comprise a deleterious allele that is homozygous in the organism. In some embodiments, the prioritized genetic variants comprise a target for gene editing. In some embodiments, the prioritized genetic variants comprise a deleterious allele homozygous in the organism that is used as a target for gene editing. In some embodiments, the gene editing is achieved by a zinc finger nuclease (ZFN) system, a transcription activator-like effector nuclease (TALEN) system, or a clustered regularly interspersed short palindromic repeats (CRISPR) system.

### DESCRIPTION OF THE FIGURES

The patent or application file contains at least one figure executed in color. Copies of this patent or patent application publication with color figures will be provided by the Office upon request and payment of the necessary fee.

FIG. 1 shows an alignment of the nucleotide sequences encoding hypothetical proteins from five organisms: sorghum (Sorghum bicolor), maize (Zea mays) inbred line B73, maize (Zea mays) inbred line Mo17, foxtail millet (Setaria italic), and big bluestem grass (Andropogon gerardi), corresponding to SEQ ID NOS. 1-5.

FIG. 2 shows an alignment of the polypeptide sequences of hypothetical proteins from five organisms: sorghum (Sorghum bicolor), maize (Zea mays) inbred line B73, maize (Zea mays) inbred line Mo17, foxtail millet (Setaria italic), and big bluestem grass (Andropogon gerardi), corresponding to SEQ ID NOS. 6-10.

FIG. 3 shows the distribution of transcript splicing ratios for gene GRMZM2G009593 in a maize population. The orange dotted line indicates the maize line B104. The blue dotted lines indicate the 95% confidence interval.

FIG. 4 shows the different biological pathways identified in three sets of hybrids, where Path_12783 is commonly shared by all three sets, suggesting that Path_12783 is likely a biological pathway underlying hybrid performance in maize.

FIG. 5 shows the outlier statuses of an endophenotype (e.g. gene expression or protein abundance) of a gene possessing a putative deleterious allele in its coding sequence (CDS) and its neighboring genes in a gene network in an organism.

FIG. 6 shows that after a genetic perturbation is made to remove the putative deleterious mutation of the gene, the outlier statuses of the endophenotype (e.g. gene expression or protein abundance) of the gene and its neighboring genes in a gene network are corrected, suggesting the putative deleterious mutation is likely to be deleterious and has a negative impact on fitness and performance of the organism.

FIG. 7 shows a scheme of how to use allele specific expression (ASE) and/or allele specific splicing in a hybrid away from the putatively deleterious allele to test hypotheses regarding which genetic variants are likely deleterious and to acquire evidence that a specific allele is in fact likely deleterious.

FIG. 8 shows a flowchart of the processes for using statistical models, feedback from endophenotypic assays, and machine learning to assess genetic variants.

FIG. 9 shows the correlation between allelic expression and predicted effect on fitness of genetic variants in expressed genes from 23 different tissues or developmental stages in hybrids derived from B73 crossed to Mo17, wherein a strong allelic expression bias is found in genes where one inbred parent in a hybrid pairing contains an allele that is dramatically more deleterious compared to the most deleterious allele in the same gene in the other inbred parent. X-axis shows the difference in Protein Variation Effect Analyzer (PROVEAN) score between two parental alleles in a hybrid, representing the predicted effect on fitness of each variant in expressed genes. Y-axis shows the biased allele expression.

FIG. 10 shows the correlation between allelic expression and predicted effect on fitness of genetic variants in expressed genes from 23 different tissues or developmental stages in hybrids derived from B73 crossed to Mo17, wherein when there is a big difference between a hybrid's two parental allele fitness levels the deleterious allele is either avoided for expression or overexpressed. X-axis shows the difference in Protein Variation Effect Analyzer (PROVEAN) score between two parental alleles in a hybrid, representing the predicted effect on fitness of each variant in expressed genes. Y-axis shows the derived allele ratio.

FIG. 11 shows the null hypothesis model and the working model illustrating that it is the absolute magnitude of the expression imbalance between the two parental alleles, rather than the direction of the imbalance, that serves as an indicator of a gene possessing a deleterious allele.

FIGS. 12A-12D show alignment of the coding sequences (CDS) and protein sequences (SEQ ID NOs. 11-18) of two genes, Zm00001d025973 and Zm00001d025973, which exhibit strongly biased allele specific expression (ASE) in hybrids that is potentially driven by deleterious allele from one copy of the inbred parent.

FIG. 13 shows the endophenotypes in the form of gene expression in germinating maize kernel roots of the corresponding expression network partners of the Zm00001d047446 gene in the maize line B104, which possesses the derived putatively deleterious allele S277P (SEQ ID NO. 19), are displayed as vertical dash lines relative to the population distribution.

FIG. 14 shows the endophenotypes in the form of gene expression in germinating maize kernel roots of the corresponding expression network partners of the Zm00001d002452 gene in the maize line B104, which possesses the derived putatively deleterious allele P37L (SEQ ID NO. 20), are displayed as vertical dash lines relative to the population distribution.

FIG. 15 shows the endophenotypes in the form of gene expression in germinating maize kernel roots of the corresponding expression network partners of the Zm00001d016008 gene in the maize line B104, which possesses the derived putatively deleterious allele V232I (SEQ ID NO. 21), are displayed as vertical dash lines relative to the population distribution.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Genetic variants refer to the alternate sequences of DNA at a specific region of the genome between organisms, or the alternate amino acid sequences encoded thereby, which serve as the source and targets for genetic improvement of organisms. However, the number of genetic variants for a given genome can be enormous, and the effect of a genetic variant can be either neutral, favorable, or deleterious to the fitness and performance of an organism. Therefore, to achieve efficient and effective genetic improvement of an organism, genetic variants need to be assessed for their effects such that subsequent breeding effort can be prioritized in selecting for or against such variants, or modifying thereof.

The present invention is based, at least in part, on the surprising results that increased effectiveness and efficiency of assessing genetic variants are observed by assessing the endophenotype of a particular variant and updating a model based upon the results. Accordingly, provided herein are methods for assessing genetic variants for use as targets in genetically improving organisms and in human genetics and medicine. Also provided herein are systems for implementing such methods, as well as computer-readable storage media storing instructions for performing such methods.

Accordingly, in one aspect, provided herein is a method for prioritizing genetic variants, comprising: a) providing a plurality of genetic variants in the genome of an organism; b) predicting the effects of the genetic variants on the performance of the organism using an endophenotype; and c) prioritizing the genetic variants based on the magnitudes of the predicted effects on the performance of the organism.

As used herein, the terms “genetic variant” and “variant” refer to a nucleotide or polypeptide sequence that differ from a reference sequence for a given region. For example, a genetic variant may comprise a deletion, substitution, or insertion of one or more nucleotides or amino acids encoded thereof. When the reference sequence refers to a normal or wild-type sequence, a genetic variant may also be referred to as a “mutation” and an organism having such mutation as a “mutant.” When it is used in the context of an alternative form of a sequence, especially that of a gene in a population, a genetic variant may also be referred to as an “allele.” Accordingly, in some embodiments, the genetic variant of the present disclosure is allele. In some embodiments, the genetic variant is a mutation.

Various types of genetic variants may be used with the methods of the present disclosure, which include, for example, frameshift, stop gained, start lost, splice acceptor, splice donor, stop lost, inframe indel, missense, splice region, synonymous, and copy number variants. Non-limiting types of copy number variants include deletions and duplications. The genetic variants in the present disclosure may be provided by comparing different sequences at a given region. Methods and techniques of sequencing and sequence alignment are known in the art. See e.g., Adams et al., eds. Automated DNA sequencing and analysis. Elsevier, 2012, Franca et al., Quarterly reviews of biophysics, 35(2), 169-200, and Rosenberg, M. S. ed., 2009. Sequence alignment: methods, models, concepts, and strategies. Univ of California Press.

In some embodiments, the genetic variants of the present invention are those that exhibit epistasis. As used herein, the term “epistasis” (also known as “epistatic interaction” or “epistatic relationship”) refers to an interaction between variants of within or between genetic sequences, including, for example, genetic variants, where the presence of one genetic variant has an effect conditional on the presence of one or more additional genetic variants. Epistasis occurs both within and between molecules. Epistatic sequences may refer to alleles of a gene, genetic variants (e.g., mutations) of a gene, or sequences (e.g., genes, genetic variants) within a gene network or within a genome. Epistasis may be of various types, including, for example, dominant, recessive, complementary, compensatory, and polymeric interaction. A compensatory secondary genetic variant, for example, exhibits a compensatory epistatic interaction with a primary genetic variant. As used herein, a “compensatory” or “compensating” effect refers to a counteracting, offsetting, mitigating, and/or opposing effect. For example, relevant to a primary genetic variant, a “compensatory” or “compensating” secondary genetic variant would have a “compensatory effect” that counteracts, offsets, mitigates, and/or opposes the effect of the primary genetic variant. A compensatory secondary genetic variant may be within the same gene or gene product (e.g., polypeptide) as the primary genetic variant, i.e., a cis-acting compensatory genetic variant. A compensatory secondary genetic variant may be in a different gene or gene product (e.g., polypeptide) as the primary genetic variant, i.e., a trans-acting compensatory genetic variant. In some embodiments, the trans-acting compensatory genetic variant is within the same gene network as the primary genetic variant.

In some embodiments, the effect of a genetic variant may be represented in a numerical or mathematical form, such as an effect score. The terms “effect score” and “fitness score” refer to a representation of the effect of a variant relative to a reference or wild-type sequence. The representation may be interpretable to humans and/or machines.

The effect of a genetic variant may also refer to a value or score from a statistical model or test, including for example, a P value from a likelihood ratio test (Knudsen, B. and Miyamoto, M. M., 2001. A likelihood ratio test for evolutionary rate shifts and functional divergence among proteins. Proceedings of the National Academy of Sciences, 98(25), pp. 14512-14517), a SIFT score (Ng, P. C. and Henikoff, S., 2003. SIFT: Predicting amino acid changes that affect protein function. Nucleic acids research, 31(13), pp. 3812-3814), and a PROVEAN score (Choi, Y., Sims, G. E., Murphy, S., Miller, J. R. and Chan, A. P., 2012. Predicting the functional effect of amino acid substitutions and indels. PloS one, 7(10), p.e46688). In some embodiments, SIFT is performed with proteins having at least 80%, at least 85%, at least 90% or at least 95% identity. In some embodiments, a genetic variant is deleterious if the SIFT score is less than 0.1, less than 0.05, or less than 0.01.

Accordingly, in one aspect, provided herein is a method for improving performance of an organism, including: a) providing a plurality of genetic variants in the genome of the organism; b) predicting the effects of the genetic variants on the performance of the organism using a statistical model; c) altering one or more of the genetic variants in the genome of the organism; d) identifying an impact of the alteration on an endophenotype of the organism; e) updating the statistical model using the identified endophenotypic impact; f) optionally repeating steps c) to e) for one or more times; g) determining the genetic variants having a predicted negative effect on the performance of the organism using the updated statistical model; and h) modifying in the genome one or more of the genetic variants having a predicted negative effect on the performance of the organism, thereby improving performance of an organism.

In another aspect, provided herein is a method for selecting an organism with improved performance in a population, including: a) providing a population of organisms; b) providing a plurality of genetic variants of the population; c) predicting the effects of the genetic variants on the performance of the organisms using a statistical model; d) altering one or more of the genetic variants in one or more of the organisms; e) identifying an impact of the alteration on an endophenotype in the one or more of the organisms; f) updating the statistical model using the identified endophenotypic impact; g) optionally repeating steps d) to f) for one or more times; h) determining the genetic variants having predicted positive effects on the performance of the organisms using the updated statistical model; and i) selecting in the population an organism comprising one or more of the genetic variants having predicted positive effects on the performance of the organisms, selecting an organism with improved performance in a population.

In yet another aspect, provided herein is a method for removing an underperforming organism from a population, including: a) providing a population of organisms; b) providing a plurality of genetic variants of the population; c) predicting the effects of the genetic variants on the performance of the organisms using a statistical model; d) altering one or more of the genetic variants in one or more of the organisms; e) identifying an impact of the alteration on an endophenotype in the one or more of the organisms; f) updating the statistical model using the identified endophenotypic impact; g) optionally repeating steps d) to f) for one or more times; h) determining the genetic variants having predicted negative effects using the updated statistical model; and i) removing from the population an organism comprising one or more of the genetic variants having predicted negative effects on the performance of the organisms, thereby removing an underperforming organism from a population.

In still another aspect, provided herein is a method for prioritizing genetic variants based on predicted effects on performance of an organism, including: a) providing a plurality of genetic variants in the genome of the organism; b) predicting the effects of the genetic variants on the performance of the organism using a statistical model; c) altering one or more of the genetic variants in the genome of the organism; d) identifying an impact of the alteration on an endophenotype of the organism; e) updating the statistical model using the identified endophenotypic impact; f) optionally repeating steps c) to e) for one or more times; and g) prioritizing the genetic variants based on the magnitudes of the predicted effects on the performance of organism using the updated statistical model.

The organism of the present invention may be any organism that is of economic and/or scientific value to humans. In some embodiments, the organism is a plant. In some embodiments, the organism is maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, or a biomass crop. In some embodiments, the organism is an animal. In some embodiments, the organism is cattle, sheep, goat, horse, pig, chicken, duck, goose, rabbit, or fish. In some embodiments, the organism is an alga, such as spirulina.

Plant genomes possess certain unique characteristics that may affect how genetic variants are identified and assessed in plants versus in other organisms, e.g., animals and humans. Without wishing to be bound by any theory, it is believed that historical genome duplication events and higher ploidy beyond diploidy in plants leading to subsequent neofunctionalization of duplicated genes may prevent certain variant prediction tools that are mainly designed for use in animals or humans from being effective in plants, given that two or more copies of a gene may accumulate mutations to reach a new function. Furthermore, reorganization of the genome and the accompanying mutagenic effects of transposable elements in plant genomes leads to diversity which is greater than that in animals and humans, and these two impacts of transposable elements may obscure the signal which points to which diversity is likely functional and deleterious.

The performance of the present invention may be any phenotype, quality, or trait of the organism. For instance, in some embodiments wherein the organism is a plant, the performance may be yield, overall fitness, biomass, photosynthetic efficiency, nutrient use efficiency, heat tolerance, drought tolerance, herbicide tolerance, disease resistance. In some embodiments, the performance is yield performance in maize. “Yield performance” refers to the total amount of harvestable material, e.g. grain or forage, obtained in a typical field performance trial. In some embodiments wherein the organism is an animal, the performance may be growth rate, feed use efficiency, meat yield, meat quality, milk yield, milk quality, egg yield, egg quality, wool yield, or wool quality. In some embodiments, the performance is a quantitative trait controlled by multiple loci in the genome of the organism.

In some embodiments, the prioritized genetic variants of the present disclosure may be used as targets in precision medicine. As used herein, the terms “personalized medicine,” “individualized medicine,” and “precision medicine” refer to the tailoring of medical procedures to the individual characteristics of each patient, based on the patient's unique molecular and genetic profile that make the patient predisposed or susceptible to certain diseases. A medical procedure may be prognosis, diagnosis, treatment, intervention, or prevention.

Accordingly, in one aspect, provided herein is a method for prioritizing genetic variants for use in a medical procedure, comprising: a) providing a plurality of genetic variants in a human genome; b) predicting the effects of the genetic variants using a statistical model; c) altering one or more of the genetic variants; d) identifying an impact of the alteration on an endophenotype; e) updating the statistical model using the identified endophenotypic impact; f) optionally repeating steps c) to e) for one or more times; g) prioritizing the genetic variants based on the magnitudes of the predicted effects using the updated statistical model; and h) using the prioritized genetic variants in a medical procedure. In some embodiments, the medical procedure is prognosis, diagnosis, treatment, intervention, or prevention.

In some embodiments, provided herein is a method of treatment, comprising: a) providing a plurality of genetic variants in the genome of a patient; b) predicting the effects of the genetic variants using a statistical model; c) altering one or more of the genetic variants; d) identifying an impact of the alteration on an endophenotype; e) updating the statistical model using the identified endophenotypic impact; f) optionally repeating steps c) to e) for one or more times; g) prioritizing the genetic variants based on the magnitudes of the predicted effects using the updated statistical model; h) selecting one or more medical treatments specific to the patient based on the prioritized genetic variants; and i) administering the one or more medical treatments to the patient.

The genetic variants in the present invention may be provided by comparing sequences between genomes. Methods and techniques of sequencing and sequence alignment are known in the art. See e.g. Adams et al., eds. Automated DNA sequencing and analysis. Elsevier, 2012, Franca et al., Quarterly reviews of biophysics, 35(2), 169-200, and Rosenberg, M. S. ed., 2009. Sequence alignment: methods, models, concepts, and strategies. Univ of California Press. In certain variations, the genetic variants that are associated with performance of the organism are provided. In some embodiments, the genetic variants may be identified by a linkage study. In some embodiments, the genetic variants may be identified by an association study. In some embodiments, the association study is a genome-wide association study (GWAS) or a transcriptome-wide association study (TWAS).

Statistical models and machine learning have been used in predicting effects of genetic variants in plant and animal breeding and human medicine. Methods and techniques of statistical modeling are known in the art. See e.g. Varshney, et al. Trends in biotechnology, 2009; 27(9), 522-530, Cardoso et al. Front Bioeng Biotechnol. 2015; 3:13, and Ho et al. Frontiers in Genetics, 2019; 10. The statistical model of the present invention may be any statistical model that associates the genetic variants with the performance of the organism. Accordingly, in some embodiments, the statistical model may be a linear regression model, a logistic regression model, a ridge regression model, a lasso regression model, an elastic net regression model, a decision tree model, a gradient boosted tree model, a neural network model, or a support vector machine (SVM) model.

By way of example, putatively deleterious alleles and their impacts on yield performance may be predicted using sequential natural language deep learning models. As used herein, the term “language model,” which may refer to either a “sequential language model” or a “masked language model” refers to a machine learning method that interprets, predicts, and/or generates sequential data. At a high level, a sequential language model takes in a sequence of inputs, examines each element of the sequence, and predicts the next element of the sequence. Similarly, a masked language model takes in a sequence of inputs, a random subset of which have their ground truth masked or obscured from the perspective of the model, and predicts those masked elements. In some embodiments, the language model is a mathematical representation of the frequency and order with which specific monomeric units or gaps occur in a set of polymers, e.g., amino acid residues in a polypeptide sequence. The mathematical representation can include a probability of a given monomer occurring at a position in the sequence. In some embodiments, the language model predicts what specific monomer comes next in a sequence of different monomers—a process known as “next token prediction.” In some embodiments, the language model predicts what specific monomer should fill in a missing space in a sequence of different monomers—a process known as “masked token prediction.” A probability of a given monomer occurring at a position in the sequence model can be independent of other positions or can depend on the occupancy at any or all other positions in the sequence model. An example of a position independent model is a Hidden Markov Model. In some embodiments, the language model is configured to output a set of semantic features. These models uniquely permit the prediction of an allele's impact when it is present in combination with secondary or in higher order combination with other putatively deleterious alleles which may in fact be compensatory for the impact of the focal mutation, rendering it non deleterious. The correct prediction of these compensations through the use of sequential natural language models reduces false positive and false negative misprioritization of alleles which in turn leads to loss rather than gain of yield performance after editing such a false positive nomination of the deleterious allele.

The model of the present disclosure may be trained in various suitable ways. In some embodiments, the model is trained by: a) a pre-training task, comprising: 1) receiving a pre-training dataset comprising a plurality of batches of naturally occurring sequences; 2) inputting each batch of sequences into a sequential language model, wherein the model is configured to output a pre-training set of semantic features; 3) automatically updating the sequential language model after each batch; b) optionally, a fine-tuning task, comprising: 1) receiving a fine-tuning dataset comprising a plurality of batches of naturally occurring sequences, wherein the fine-tuning dataset is a subset of the pre-training dataset, or a set of sequences that are related to the pre-training dataset by common ancestry, homology, or multiple sequence alignment; 2) inputting each batch of sequences into the sequential language model, wherein the model is configured to output a fine-tuning set of semantic features; and 3) automatically updating the sequential language model after each batch; and c) a transfer learning task, comprising: 1) receiving a final training dataset comprising labeled sequences mapped to effects; and 2) training a neural network model based on the final training dataset, wherein the neural network model is configured to receive data corresponding to the pre-training set of semantic features and/or the fine-tuning set of semantic features, and output one or more effect scores.

The genetic variants of the present invention may be assessed, weighted, or prioritized by a statistical model based on one or more criteria. Examples of the criteria include, but are not limited to, evolutionary conservation (See e.g. Chun and Fay (2009) Genome Res. 19: 1553-1561 and Rodgers-Melnick et al (2015) PNAS 112: 3823-3828), functional impact of amino acid change (See e.g. Ng et al (2003) NAR 31:3812-3814 and Adzhubei et al (2010) Nat Methods 7:248-249), functional impact of protein conformation and/or stability (See e.g. Rosetta, a computational protein design platform from Cyrus Bio Inc.), adjacency to selective sweep regions (See e.g. Hufford et al (2012) Nat gen 44: 808-813), and outlier status of an endophenotype (See e.g. Zhao et al (2016) AJHG 98, 299-309). In some embodiments, the evolutionary conservation is determined by sequence alignment in a genic or an intergenic region. In some embodiments, the functional impact of amino acid change is weighted according to the blocks substitution matrix (BLOSUM). In some embodiments, the functional impact of protein conformation and/or stability is determined by a Monte Carlo search for minimal free energy. In some embodiments, the functional impact of protein conformation and/or stability is predicted by learning a representation of amino acid order from existing proteins in higher dimensional space. In some embodiments, the selective sweep region is determined by a decrease of pairwise nucleotide diversity π or linkage disequilibrium relative to the rest of the genome. In some embodiments that may be combined with any of the preceding embodiments, the feature is a numeric or categorical value associated with a specific allele at a genomic locus.

In some embodiments, the alteration/perturbation of the genetic variants is achieved by genome editing. As used herein, the term “genome editing” or “gene editing” refers to the process of altering the target genomic DNA sequence by inserting, replacing, or removing one or more nucleotides. Genome editing may be accomplished by using nucleases, which create specific double-strand breaks (DSBs) at desired locations in the genome, and harness the cell's endogenous mechanisms to repair the induced break by homology-directed repair (HDR) (e.g., homologous recombination) or by non-homologous end joining (NHEJ). Any suitable nuclease may be introduced into a cell to induce genome editing of a target DNA sequence including, but not limited to, clustered regularly interspersed short palindromic repeats (CRISPR)-associated protein (Cas, e.g. Cas9 and Cas12a) nucleases, zinc finger nucleases (ZFNs, e.g. FokI), transcription activator-like effector nucleases (TALENs, e.g. TALEs), meganucleases, and variants thereof (Shukla et al. (2009) Nature 459: 437-441; Townsend et al (2009) Nature 459: 442-445). Accordingly, in some embodiments of the present invention, the genome editing is achieved by a clustered regularly interspersed short palindromic repeats (CRISPR) system, a transcription activator-like effector nuclease (TALEN) system, or a zinc finger nuclease (ZFN) system.

In some embodiments, the type of genome editing is base editing. As used herein, the term “base editing” refers to a base mutation (substitution, deletion or addition) that causes point mutations in a target site within a target gene, with a few bases (one or two). Various base editors are known in the art, and may have various approximate editing windows. See e.g., Rees, H. A. and Liu, D. R., 2018. Base editing: precision chemistry on the genome and transcriptome of living cells. Nature reviews genetics, 19(12), pp. 770-788; Molla, K. A. and Yang, Y., 2019. CRISPR/Cas-mediated base editing: technical considerations and practical applications. Trends in biotechnology, 37(10), pp. 1121-1142; and Mishra, R., Joshi, R. K. and Zhao, K., 2020. Base editing in crops: current advances, limitations and future implications. Plant Biotechnology Journal, 18(1), pp. 20-31. Accordingly, in some embodiments, the editing window is from 5-10 bp. In some embodiments, the editing window is from 5-15 bp. In some embodiments, the editing window is from 5-20 bp. In some embodiments, the editing window is from 5-25 bp. In some embodiments, the editing window is from 5-30 bp. In some embodiments, the editing window is from 5-35 bp. In some embodiments, the editing window is from 5-40 bp. In some embodiments, the editing window is from 5-45 bp. In some embodiments, the editing window is from 5-50 bp. In some embodiments, the editing window is from 10-20 bp. In some embodiments, the editing window is from 10-30 bp. In some embodiments, the editing window is from 10-40 bp. In some embodiments, the editing window is from 10-50 bp.

In yet some other embodiments, the alteration/perturbation of the genetic variants is achieved by creation of novel haplotype combinations from genetic recombination during meiosis in the course of breeding with the aim of increasing the numbers of favorable alleles which are stacked together and inherited together as part of a haplotype. The presence of individual mutations and their abundance can be assessed by genotyping.

In some aspects of the present invention, the method for selecting an organism with improved performance in a population may be used for genomic selection. In some aspects of the present invention, the prioritized genetic variants may be used for genomic selection. Genomic selection (GS) estimates marker effects across the whole genome on the target population based on a prediction model developed in the training population. Methods and techniques of GS is known in the art. See e.g. Jannink, et al. Briefings in functional genomics, 2010: 9(2), 166-177, Goddard, et al. Journal of Animal breeding and Genetics 2007:124 (6), 323-330, and Desta and Ortiz. Trends in plant science 2014:19(9), 592-601.

As used herein, the term “endophenotype” refers to a quantifiable phenotype at the sub-organismal level that can be measured by a biochemical, gene expression, or protein level assay, or by a visual feature measured at the sub-organismal level, e.g., via microscopy. In some embodiments, the endophenotype is an intermediate quantitative phenotype that is biologically relevant to, associated with, or predicative of a phenotype at the organism level, such as yield performance or overall fitness. Endophenotypes can be readily measured in cells, tissue, or young organisms that serve as a proxy to quickly determine which genetic variants are more likely to have an impact on a terminal phenotype, such as yield performance or overall fitness. Examples of endophenotypes include, but are not limited to, messenger RNA (mRNA) abundance, gene transcript splicing ratio, protein abundance, micro RNA (miRNA) or small RNA (siRNA) abundance, translational efficiency, ribosome occupancy, protein modification, metabolite abundance, and allele specific expression (ASE). Endophenotypes may be associated with a genetic variant that is physically proximal or proximal within a gene network.

In some embodiments, mRNA abundance (gene expression) is affected by a genetic variant if expression is altered at least 2, at least 3, at least 4 or at least 5 fold.

In certain aspects, provided herein is an organism with improved performance produced or selected by any one of the methods disclosed in the present invention.

In certain other aspects, provided herein is a computer-implemented method for assessing genetic variants for use in genetic improvement of an organism, including: a) receiving a dataset comprising a plurality of genetic variants of the organism; and b) performing a prediction of the effects of the genetic variants using a statistical model comprising one or more initial rules that associate the genetic variants with performance of the organism. In some embodiments, the method further includes updating the statistical model with one or more new rules, wherein the one or more new rules are based on data generated from an endophenotype, wherein the endophenotype is a quantifiable phenotype at the sub-organismal level that can be measured by a biochemical, gene expression, or protein level assay, or visually via microscopy.

In yet certain other aspects, provided herein is a computer-readable storage medium storing computer-executable instructions, including: a) instructions for applying a statistical model to a dataset, wherein the dataset comprises a plurality of genetic variants of an organism, and wherein the statistical model comprises one or more initial rules that associate the genetic variants with performance of the organism; and b) instructions for calculating an effect value related to the performance of the organism for each of the genetic variants. In some embodiments, the computer-readable storage medium further includes instructions for updating the statistical model with at least one new rule, wherein at least one new rule is based on data generated from an endophenotype, wherein the endophenotype is a quantifiable phenotype at the sub-organismal level that can be measured by a biochemical, gene expression, or protein level assay, or visually via microscopy. In some embodiments, the computer-readable storage medium is a solid-state device, a hard disk, a CD-ROM, or other non-volatile computer-readable storage medium.

In still certain other aspects, provided herein is a system (e.g. a computer system) for assessing genetic variants for use in genetic improvement of an organism, including: a) a computer-readable storage medium storing a database comprising a plurality of genetic variants of the organism; b) a computer-readable storage medium storing computer-executable instructions, including: i) instructions for applying a statistical model to the dataset, wherein the statistical model comprises one or more initial rules that associate the genetic variants with performance of the organism; and ii) instructions for calculating an effect value related to the performance of the organism for each of the genetic variants; and c) a processor configured to execute the computer-executable instructions stored in the computer-readable storage medium. In some embodiments, the computer-readable storage medium further includes instructions for updating the statistical model with one or more new rules, wherein the one or more new rules are based on data generated from an endophenotype, wherein the endophenotype is a quantifiable phenotype at the sub-organismal level that can be measured by a biochemical, gene expression, or protein level assay, or visually via microscopy. In some embodiments, the system may be a server computer, a client computer, a personal computer, a user device, a tablet PC, a laptop computer, a personal digital assistant, a cellular telephone, or any machine capable of executing a set of instructions, sequential or otherwise, that specify actions to be taken by that machine. In some embodiments, the system may further include keyboard and pointing devices, touch devices, display devices, and network devices.

In some embodiments that may be combined with any of the preceding embodiments, the statistical model is a linear regression model, a logistic regression model, a ridge regression model, a lasso regression model, an elastic net regression model, a decision tree model, a gradient boosted tree model, a neural network model, or a support vector machine (SVM) model.

In some embodiments that may be combined with any of the preceding embodiments, the one or more initial rules or the one or more new rules comprise evolutionary conservation, functional impact of amino acid change, functional impact of protein conformation and/or stability, adjacency to selective sweep regions, outlier status of an endophenotype associated with a genetic variant that is physically proximal or proximal within a gene network, or a combination thereof.

In some embodiments that may be combined with any of the preceding embodiments, the endophenotype is messenger RNA (mRNA) abundance, gene transcript splicing ratio, protein abundance, micro RNA (miRNA) or small RNA (siRNA) abundance, translational efficiency, ribosome occupancy, protein modification, metabolite abundance, allele specific expression (ASE), or a combination thereof.

### EXAMPLES

The following examples are offered to illustrate provided embodiments and are not intended to limit the scope of the present disclosure.

### Example 1: Prioritizing Genetic Variants Based on Evolutionary Conservation

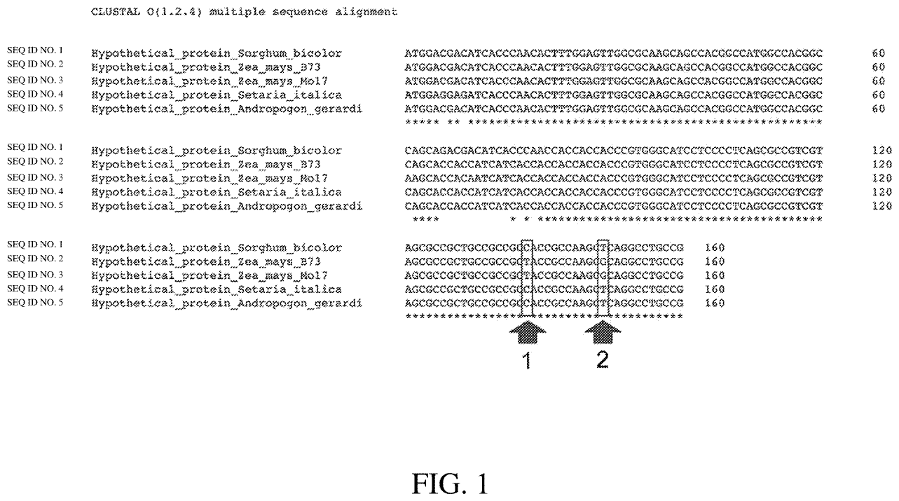

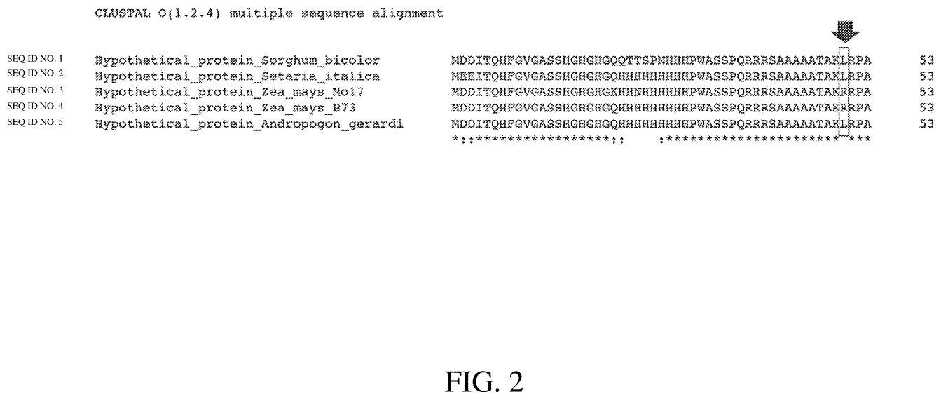

The genomic sequences of pairs of inbred maize lines known to have good combining ability for making hybrids are compared with those of five or more related panicoid species. This allows for the detection of variants that deviate in maize from evolutionary consensus and are not complemented in the specific hybrid created by the pair of lines in question. Accordingly, syntenic chromosomal fragments are aligned, and regions of high sequence conservation are parsed. The aligned sequences in which three or more of the related panicoid species' genomes can be aligned with maize are analyzed for polymorphisms present only in one or more of the maize sequences but infrequent in the related panicoid species. The genetic variants shared by the two maize inbred lines (B73 and Mo17) are then prioritized for subsequent editing and/or selection to improve yield performance of the hybrid. Sequences are evaluated using this method in any genomic locations where multiple sequence alignment could be performed including genic and intergenic regions. FIG. 1 and FIG. 2 show alignment of the nucleotide sequences encoding, and the polypeptide sequences of, respectively, a hypothetical protein from five organisms: sorghum (Sorghum bicolor), maize (Zea mays) inbred line B73, maize (Zea mays) inbred line Mo17, foxtail millet (Setaria italic), and big bluestem grass (Andropogon gerardi), where mutation 1 is a synonymous mutation of alanine to alanine and mutation 2 causes an amino acid change from leucine to arginine.

In some instances, measurements of conservation based on statistical tests may be used as criteria to further assist in prioritizing genetic variants. Non-limiting examples of such statistical tests include the Genomic Evolutionary Rate Profiling framework (GERP; see e.g., Davydov, E. V., Goode, D. L., Sirota, M., Cooper, G. M., Sidow, A. and Batzoglou, S., 2010. Identifying a high fraction of the human genome to be under selective constraint using GERP++. PLoS Comput Biol, 6(12), p.e1001025) and likelihood ratio tests (LRTs; see e.g., Knudsen, B. and Miyamoto, M. M., 2001. A likelihood ratio test for evolutionary rate shifts and functional divergence among proteins. Proceedings of the National Academy of Sciences, 98(25), pp. 14512-14517.).

### Example 2: Prioritizing Genetic Variants Based on Functional Impact of Amino Acid Change

Genetic variants identified from multiple sequence alignments in Example 1 that are in protein coding sequences and lead to a predictable amino acid change receive a weight (e.g. substitution score) between 0 and 1 based on the severity of the amino acid change. A substitution score of 0 denotes the amino acid change is not anticipated to have an effect on the protein, such as in the case of substituting a small non polar glycine for a similar small non-polar amino acid like alanine. A substitution score of 1 denotes the impact of a change such as from a small non-polar amino acid like a glycine to a large polar or aromatic amino acid like a tryptophan, with specific score further altered by local sequence context. These weights are based on known properties of the amino acid, for example as described by a BLOSUM matrix. For mutation 2 in FIG. 1, the non-maize species possess a leucine (a hydrophobic amino acid) and the two maize lines possess an arginine (an amino acid with a charged basic side chain); therefore, mutation 2 is upweighted. Synonymous mutations that do not alter the protein sequence but change the codon are weighted with a value that is inversely proportional to their frequency in maize, such that a base change away from the evolutionary consensus which also switches the codon to one which is rarer in maize receives a greater weight. For mutation 1 in FIG. 1 which is synonymous, the codon GCC is changed to GCT. GCC has a frequency of 31% and GCT has a frequency of 21% in maize; therefore mutation 1 receives an elevated codon frequency weight. Specifically, the mutation receives a weight equal to:

w=(1/x_{q})*(1/(1+y_{q}))

Where:
x_{q}=frequency of the codon q in the corn genome
y_{q}=number of additional codons which encode the amino acid encoded by codon q

Therefore, when a synonymous mutation leads to a codon of an amino acid which is encoded by 4 codons and the new codon resulting from the mutation has a frequency of 25% the weight will be 1/0.25*1/(1+3)=1. In other words, synonymous mutations to codons which are frequent are not upweighted. However, for mutation 2 in FIG. 1 which changes the GCC (freq 33.7%) to GCT (freq 22.8%) the mutation is upweighted to 1.10=(1/xq)*(1/(1+yq))=(1/0.228)*(1/(1+3)) in this category. If the mutation differs from the evolutionary consensus determined by aligning against related panicoid grasses, but results in changing from a rare codon to a more common codon in maize, it receives a lower weight in this category because it is less likely functionally deleterious.

In some instances, computational algorithms and tools for predicting functional effect of amino acid substitution may be used as criteria to further assist in prioritizing genetic variants. Non-limiting examples of such computational algorithms and tools include Protein Variation Effect Analyzer (PROVEAN; see e.g., Choi, Y., Sims, G. E., Murphy, S., Miller, J. R. and Chan, A. P., 2012. Predicting the functional effect of amino acid substitutions and indels. PloS one, 7(10), p.e46688) and Sorting Intolerant from Tolerant (SIFT; see e.g., Ng, P. C. and Henikoff, S., 2003. SIFT: Predicting amino acid changes that affect protein function. Nucleic acids research, 31(13), pp. 3812-3814).

### Example 3: Prioritizing Genetic Variants Based on Protein Conformation and Stability

Non-synonymous genetic variants in coding sequences are also prioritized for subsequent editing and/or selection based on their predicted impact on protein conformation and protein stability. The predicted impact on protein stability or folding of a genetic variant is quantified using computational tools which calculate protein confirmation or stability. The first class of these tools relies on a Monte Carlo search of possible conformations to determine the confirmation with minimal free energy such as Rosetta (Cyrus Biotechnology, Inc). The second class is machine learning-based protein stability prediction tools, for example, UniRep (Alley et al., bioRxiv (2019): 589333), Doc2Vec (Biswas et al., bioRxiv (2018): 337154), and the method of Rives et al. (bioRxiv (2019): 622803), which are trained using an evolutionarily diverse corpus of existing protein sequences from across species. By learning a representation of amino acid order from existing proteins in higher dimensional space, predictions of the likely stability of previously unseen proteins or previously unseen mutations could be calculated. This allows for the individual genetic variants at the DNA level that have a corresponding impact on protein to be further prioritized.

### Example 4: Prioritizing Genetic Variants Based on Adjacency to Selective Sweep Regions

Genetic variants identified above also receive a weight based on adjacency to selective sweep regions where genetic diversity is reduced relative to less bottlenecked tropical lines and the lines in the panicoid relative species (Hufford et al., Nature Genetics, 2012:44(7), 808-811). Severity of selective sweep is quantified by a local drop in nucleotide diversity as can be measured by pairwise nucleotide diversity π or linkage disequilibrium relative to the rest of the genome. This filter aids in identifying the genetic variants where domestication and subsequent breeding have led to a loss of diversity and likely to have fixed the non-favorable variants segregating in the ancestral population in the two inbreds used to make the hybrid.

### Example 5: Prioritizing Genetic Variants Based on Outlier Status Severity of Endophenotypes Measured for Proximal Genes

Variants are also weighted by outlier status of an endophenotype associated with a genetic variant that is physically proximal or proximal within a gene network. Examples of endophenotypes include expression level, splicing ratio, protein abundance, translation rate, ribosome occupancy, and protein phosphorylation of the gene proximal to the variant in question. The proximity can be measured physically as a distance in cis to the variant in question or in the form of proximity within a co-expression, co-translation, co-protein abundance network. Outlier status of an endophenotype within an individual can be assessed by quantifying standard deviation (s.d.) from the mean of the population or s.d. from the measured amount in an ancestral population or set of related species. Each genetic variant receives a combined prioritization score based on the totality of the available data. The prioritization score epsilon is calculated as Y=sum (Coefficient×Weight), in which the weight is a relative value in the 0-1 scale proportional to a probability that a mutation is relevant to yield performance for every criterion being input into the model, and the coefficient is a value indicative of the relative weight of the respective criterion in the overall score.

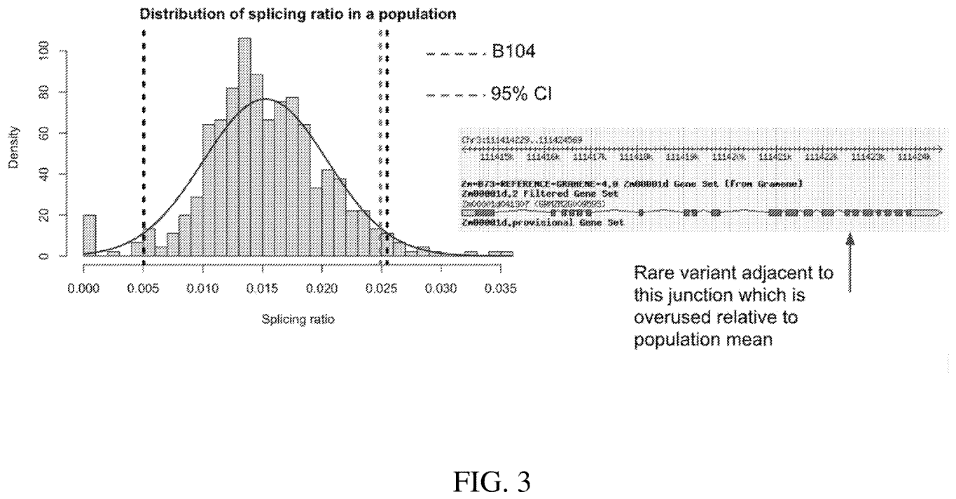

FIG. 3 shows an example of a genetic variant present in the maize line B104, which is adjacent to a splice junction that is used to a level nearly two standard deviations away from the population average. In this case the variant is annotated as being physically proximal to a splice status outlier, which therefore leads to an elevated weight in this annotation category.

### Example 6: Endophenotypes—mRNA and Protein Abundance

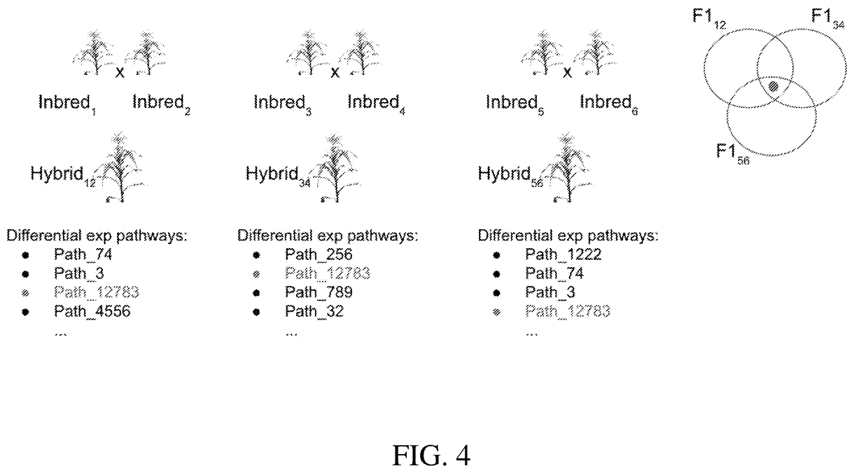

Quantification of mRNA or protein abundance of components of systems which are detected as differentially expressed between high and low performing maize plants are used to assess impacts on yield performance at the cell level. Analyses that compare the expression profiles of inbred vs. hybrid, as well as mutant vs. wild type individuals indicate specifically that the pathways in FIG. 4 and Table 1 and their component genes have utility as cell level indicators of yield performance. In FIG. 4, Path_12783 is an example of a common/shared expression signature which differentiates hybrid from inbred expression. Table 1 illustrates specific examples of such signatures determined from analysis of publicly available datasets.

TABLE 1
Biological pathways predicted to be associated with hybrid performance
in maize

| | |
|---|---|
| GO:0003735 | Structural constituent of ribosome |
| GO:0046982 | Protein heterodimerization activity |
| GO:0005506 | Iron ion binding |
| GO:0020037 | Heme binding |
| GO:0003700 | DNA binding transcription factor activity |
| GO:0005507 | Copper ion binding |
| GO:0008559 | ATPase-coupled xenobiotic transmembrane transporter |
| | activity |

Based on the evidence above and published evidence that protein turnover pathways are markers of cell level performance across species (reviewed by Goff et al., New Phytologist, 2010:189(4), 923-937), pathways and individual genes involved in translational machinery and rate, protein misfolding response, and No-go protein decay component abundance can be used as quantitatively measurable endophenotypes to assess whether a specific nominated genetic perturbation created the desired endophenotypic impact. These endophenotypes can be assayed at the level of gene expression, protein level, metabolites or with antibodies against the proteins in these pathways to detect abundance using methods like FACs or quantitative microscopy of individual cells that have been antibody-labelled.

### Example 7: Using Higher Dimensional Representations of Endophenotypes as Performance Indicators

Linear and non-linear combinations of higher dimensional encoding of expression (or other quantitative endophenotypic) values which best distinguish inbred from hybrid and mutant from wild type is used as endophenotypic readouts beyond standard GO and pathway level enrichment indicated in the section above. Embeddings are created by dimensionality reduction techniques, such as in principal coordinate analysis, principal component analysis, and stochastic neighbor embedding. Additionally, following the example of word embedding from natural language processing models including word2vec (Mikolov et al., 2013 arXiv:1310.4546), dense vector gene expression embeddings from a corpus of high dimensional expression values across individuals (40,000+ gene expression values per individual per tissue) are also created as in the first step of a neural network and are then used to distinguish between high and low performing plants based on expression. Combinations of these dimensions which best distinguish high from low performing plants are then used to evaluate the favorability of an individual genetic perturbation based on its endophenotypic consequences.

### Example 8: Using Endophenotype Outlier Status as a Performance Indicator

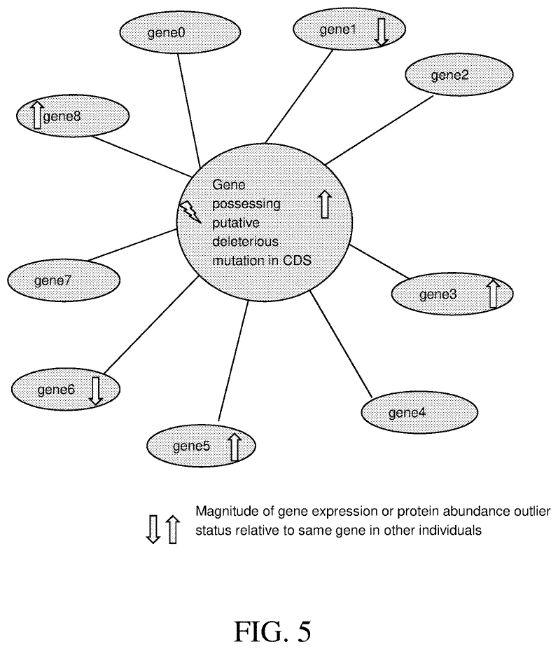

FIG. 5 shows the outlier statuses of an endophenotype (e.g. gene expression or protein abundance) of a gene possessing a putative deleterious allele in its coding sequence (CDS) and its neighboring genes in a gene network in an organism.

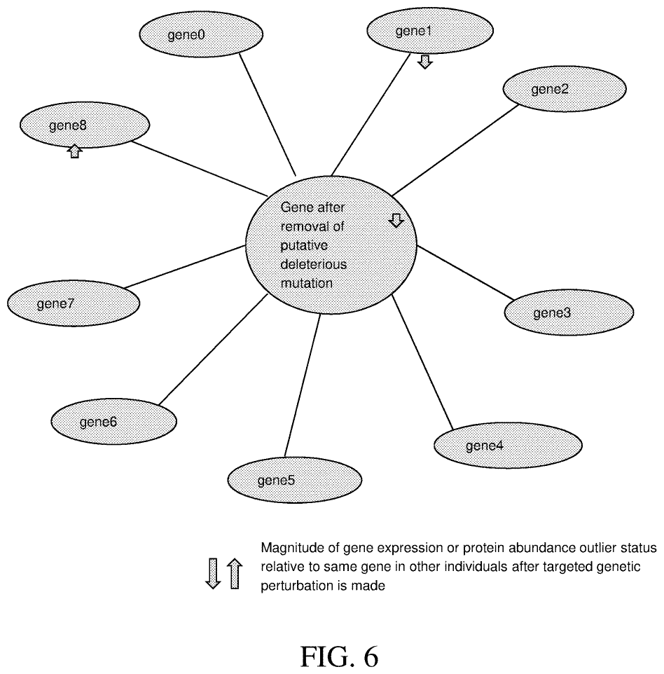

FIG. 6 shows that after a genetic perturbation is made to remove the putative deleterious mutation of the gene, the outlier statuses of the endophenotype (e.g. gene expression or protein abundance) of the gene and its neighboring genes in a gene network are corrected, suggesting the putative deleterious mutation is likely to be deleterious and has a negative impact on fitness and performance of the organism. In FIGS. 5 and 6, the length of arrow correlates to magnitude of outlier status, and the direction of arrow is the direction of outlier status.

Reduction in endophenotype outlier status can also be used as an assay for a correction to a putative deleterious cis alleles which are not harbored in the coding sequence, but rather in regulatory regions upstream, (See Kremling et al, 2018 Nature, 555(7697), 520-523, and Zhao et al., 2016 The American Journal of Human Genetics, 98(2), 299-309). Based on the aforementioned references, it is known that local abundance of putative deleterious variants associates with severe under or over-expression status of the gene downstream of the deleterious allele and that the expression dysregulation of those genes can be used to predict fitness in inbreds using penalized regression models like ridge regression or lasso. Therefore, the reduction in this outlier status can be used as evidence that the targeted alteration had the desired effect on an endophenotype and thus is likely to have a positive effect on fitness when corrected.

Reduction in endophenotype outlier status can also be used to read out the impact of a putative deleterious mutation with a predicted effect on splicing by looking for splicing outlier status instead of expression outlier status in the mutation-containing and network partner genes.

### Example 9: Endophenotype—Translational Efficiency

The ratio of expressed vs. actively translated specific alleles of mRNA sequences indicates translational efficiency which can indicate if an allele has a negative effect on translation but not expression. This allows for detection of differentially translated alleles of proteins which may be distinguished by a hybrid by differential rates of translation, but not detectable at the level of differential gene expression. This can be quantitatively assessed for each mRNA transcript by quantifying expression level with standard RNA-seq and quantifying those transcripts which are being actively translated with Ribo-Seq (Ingolia et al., 2009 Science 324: 218-223) and normalizing the latter by the former which indicates if a message is under or over-translated relative to its mRNA abundance level. mRNA messages which are detected as defective in the organism at the level of translation and thus have low translational efficiency can be used as quantitatively measurable indicators of deleterious mutation(s) in the DNA that encodes those mRNAs. As with outlier status in expression and allele specific expression, allele specific translation rate provides an endophenotypic readout which can be used both to detect likely deleterious mutations and as a readout for when they have been corrected.

### Example 10: Endophenotypes—Allele Specific Expression (ASE)

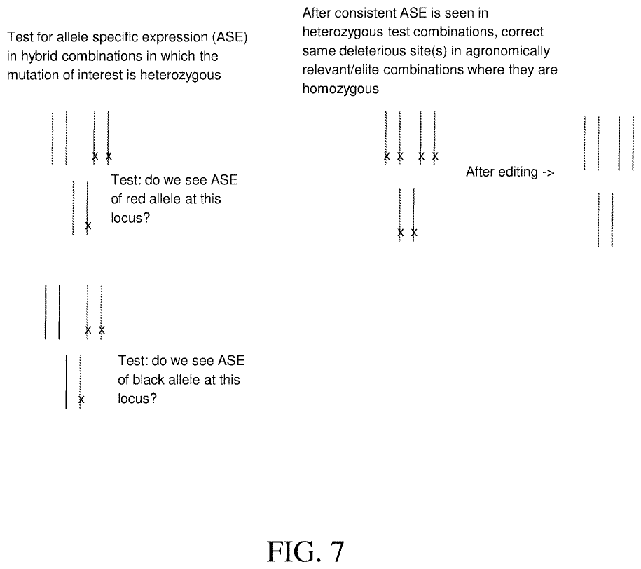

FIG. 7 shows a scheme of using allele specific expression (ASE) and/or allele specific splicing in a hybrid away from the putatively deleterious allele to test hypotheses regarding which variations are likely deleterious and to acquire evidence that a specific allele is in fact likely deleterious. Upon editing, the same expression assay can be used to assess if the putative deleterious mutation has been corrected and is thus no longer being avoided for expression. The deleterious mutations which are of greatest interest are those which are not complemented in a specific hybrid, thus a specific deleterious allele must be tested in other hybrid combinations where they are complemented (i.e. is heterozygous). ASE of the allele which is not the putatively deleterious mutation in a specific hybrid combination can be used as evidence that the mutation is in fact deleterious. This prior information of ASE in a hybrid pairing where the mutation is heterozygous can then be used to guide editing in parental pairs which share the same deleterious mutation which is not complemented in the resulting hybrid.

### Example 11: Using Statistical Models and Machine Learning to Assess Genetic Variants

Numerical and categorical features derived from the scores assigned by the computational filters described in detail in previous examples are used as input features for machine learning models which predict if a given mutation is likely to be deleterious and thus should be prioritized for editing. Input features can be numerical (e.g. the fraction of related monocotyledons species which share the alternate allele at the locus in question, or the −log 10 p-value of a GWAS association with that locus) or categorical (e.g. if a mutation leads to a nonsense or missense or synonymous mutation). Features are organized as shown in the central table in the figure below with each row representing a genetic locus with a unique chromosomal coordinate relative to a single reference genome or a pan-genome and each column representing the numerical or categorical features or higher order combination of features that serve as input to an ensemble machine learning model.

In some embodiments the ensemble machine learning model in which the above described features are used can be a regression, a logistic regression, a decision tree, a gradient boosted tree, a penalized regression method like Bayesian lasso, ridge regression or elastic net, or a support vector machine.

The machine learning model is then trained using labeled data with ‘y’ values from a subset of variants which were edited and then had the endophenotypic consequences measured (as described above in previous examples) in a cell, callus, or plantlet assays. Labeled training data is then split into a training set, a test set, and a held out validation set. Per standard practice the discipline, nested cross validation is used to train the model and assess its performance on previously unseen labeled data.

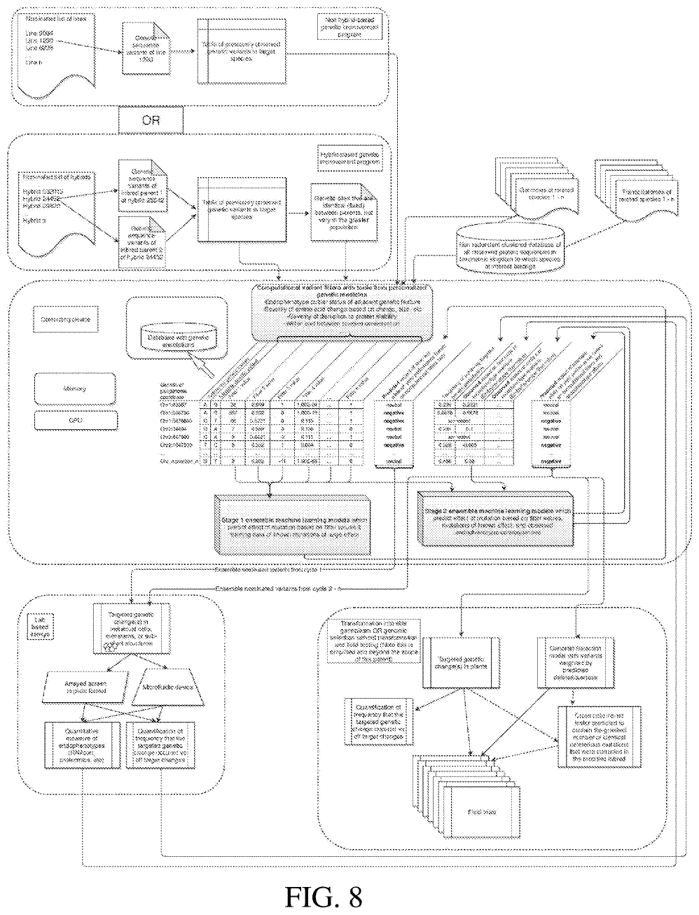

FIG. 8 shows a flowchart of the processes. Stage 1 models are trained in two ways. First, the models can be trained to maximize the combinations of features that create the strongest enrichment of putatively deleterious alleles in regions of the genome which are known to have the least amount of recombination such as in pericentromeres. Second, the stage 1 models are trained by selecting different combinations of prioritized variants using scores of the computational filters described above and calculating the variance explained by each subset of variants in a regression model. The subset which explains the most phenotypic variance in a regression are those which are tested first in the cell based assay (See Speed et al., 2012 The American Journal of Human Genetics, 91(6): 1011-1021, and Rodgers-Melnick et al., 2016 Proceedings of the National Academy of Sciences, 113(22), E3177-E3184).

The model which is trained in a supervised manner on labeled training data is then used to assess the likely impacts on yield performance of mutations which are annotated using features created in step 1) above, but which have not had an associated endophenotypic measurement taken as part of an assay. After nominating the mutations with the predicted largest and smallest effect on yield performance using the trained machine learning model, these variations are introduced into cells, callus or plantlets and endophenotypic measurements are taken. These measurements are then used to label additional variants in the table which were nominated in the last round and can then be used as training data. The model is then retrained after including the additional training instances created during the last round. The process is repeated iteratively until additional gains in accuracy reach a plateau or as resources allow.

After repeated cycles of training and validation through additional endophenotypic assays described above, the most likely deleterious variants as nominated by the machine learning model are edited in tissue that is grown into mature plants which are then grown in the field. In hybrid species like maize, loci which have the predicted deleterious mutation present and homozygous in both parents of a known high performing hybrid are carried forth for editing in tissues which will become mature plants that can be grown in the field and crossed to make hybrid seed.

### Example 12: Using Allele Specific Expression (ASE) as an Endophenotype to Identify and Prioritize Genetic Variants in Maize

This example describes the use of allele specific expression (ASE) as an endophenotype to identify and prioritize genetic variants that impact fitness in maize (Zea mays).

### Materials and Methods

To identify and prioritize genetic variants that impact fitness in maize, gene level allele specific expression (ASE) data from F1 maize hybrids made between two distinct maize inbred lines, B73 and Mo17, were obtained, and the predicted deleteriousness of the SNPs contained in each gene was analyzed. A Protein Variation Effect Analyzer (PROVEAN) score was used to quantify the fitness of each parental allele. PROVEAN scores are widely used in population genetics to decide whether an amino acid substitution or indel has an impact on the biological function of a protein. If the PROVEAN score is equal to or below a predefined threshold (e.g. −2.5), the variant is predicted to have a “deleterious” effect; otherwise, the variant is predicted to be “neutral”. See, e.g., Choi, Yongwook, and Agnes P. Chan. “PROVEAN web server: a tool to predict the functional effect of amino acid substitutions and indels.” Bioinformatics 31.16 (2015): 2745-2747.

A PROVEAN score for each of the coding sequence (CDS) SNPs as found in the maize haplotype map HapMap 3.2.1 (Bukowski, Robert, et al. “Construction of the third-generation Zea mays haplotype map.” Gigascience 7.4 (2018): gix134) was calculated to quantify the putative deleteriousness. For each gene, the minimum PROVEAN score of all derived SNPs contained in that individual (e.g. from B73 or Mo17) was used to represent that specific gene copy's fitness level in that individual. Then, the fitness difference between the two copies of the gene (one in each of the two parents) for a specific gene can be represented as the difference between the PROVEAN scores from each parental copy of the gene.

The relationship between allelic expression imbalance and the local deleterious allele burden as explained above may, for instance, be quantified mathematically as follows:

$$ω = \frac{{τ}_{jk = 1} + {τ}_{jk = 2}}{2 ( min ( {τ}_{jk = 1} , {τ}_{jk = 2} ) )} \qquad Equation 1$$

$${δ}_{j} = \vert {min}_{i in j} ( {γ}_{ijk = 1} ) - {min}_{i in j} ( {γ}_{ijk = 2} ) \vert \qquad Equation 2$$

$${ρ}_{j} = \frac{cov ( ω , δ )}{{σ}_{ω} {σ}_{δ}} \qquad Equation 3$$

i=SNP
j=Gene
k=Inbred which is source of the allele in the F1 pairing
γ=Provean_score (or other public deleteriousness assessment tool)
τ=expression level of parental allele in F1
ω=allelic expression imbalance ratio
δ=difference in fitness of both parental copies of each gene based on most deleterious SNP in each gene in each parent
ρ=pears on correlation between δ and ω
Cov=covariance
σ=standard deviation

### Results

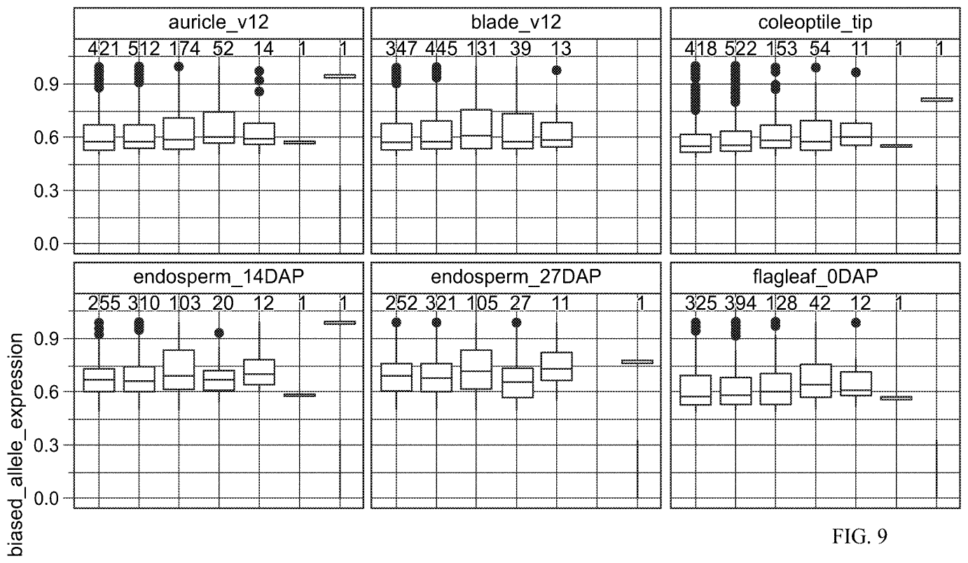

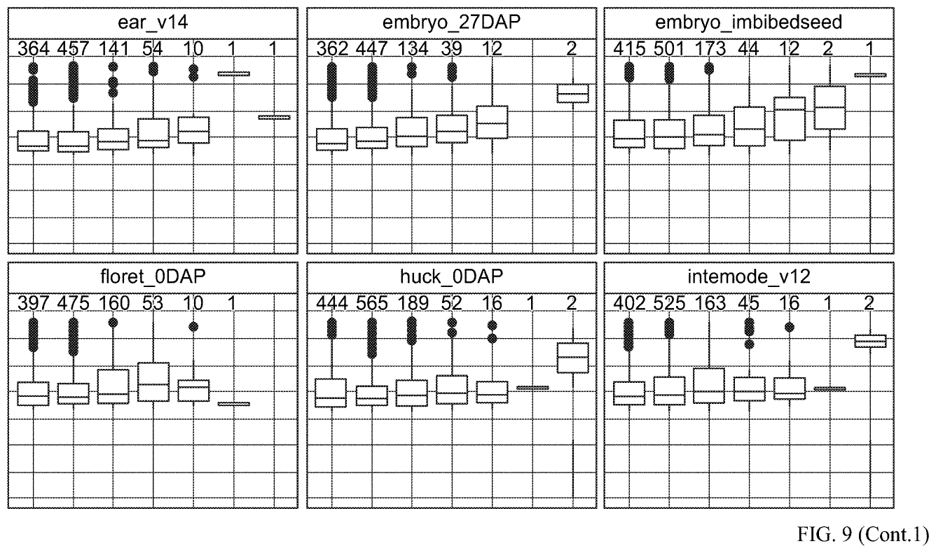

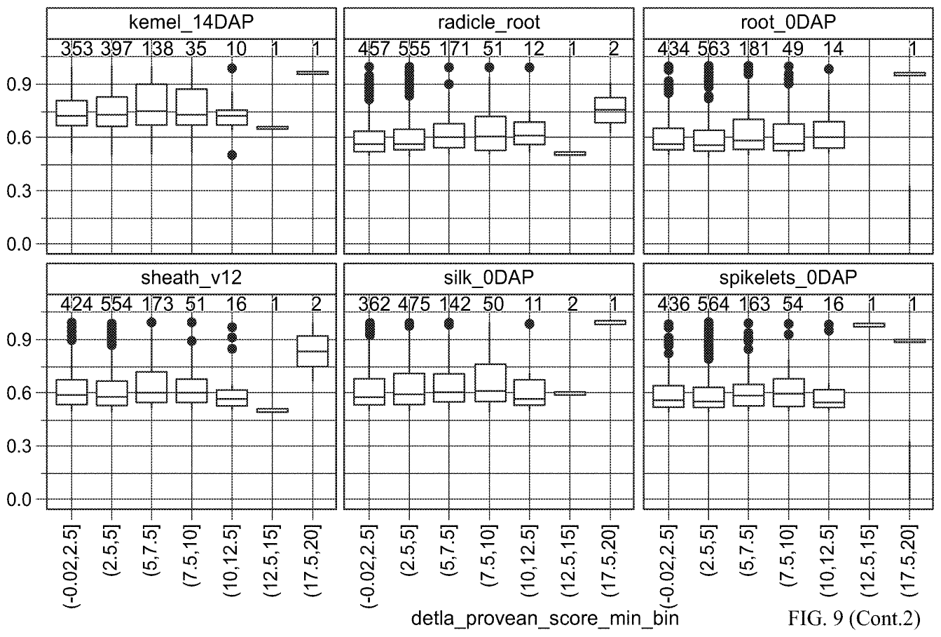

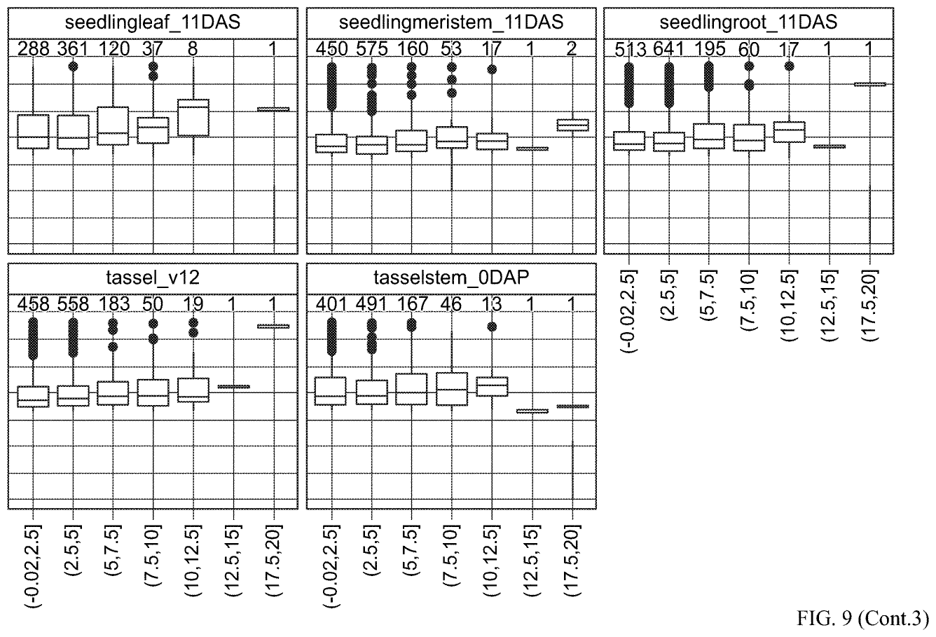

A strong allelic expression bias was found for genes where one inbred parent in a hybrid pairing contains an allele that is dramatically more deleterious compared to the most deleterious allele in the same gene in the other inbred parent. Therefore, allelic expression bias serves as an independent indicator of the presence of a deleterious allele at a variant position that differs between the parents and can be used to prioritize putative deleterious alleles by their likely severity. FIG. 9 shows such results in a hybrid derived from B73 crossed to Mo17 by checking the correlation between allelic expression and predicted effect on fitness of each variant in expressed genes from 23 different tissues or developmental stages.

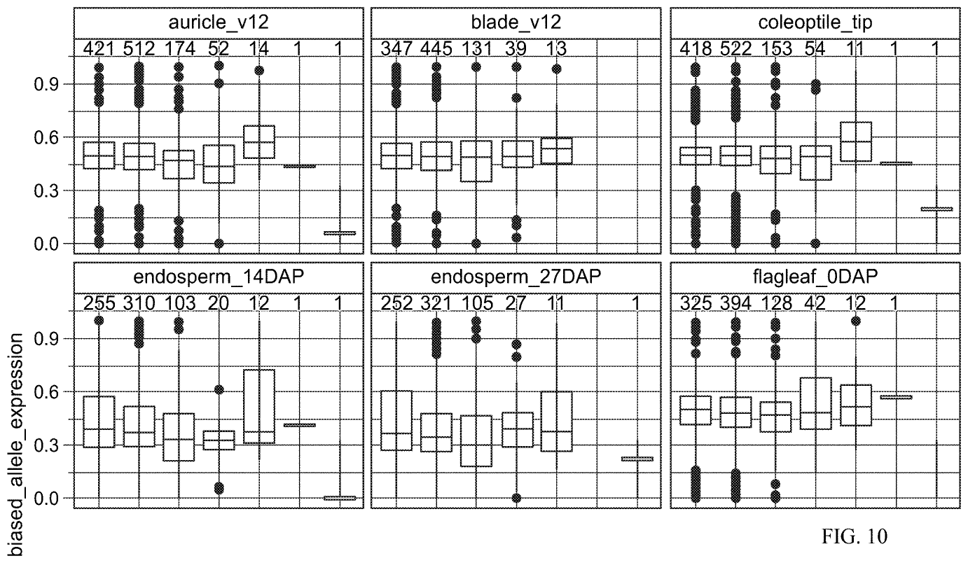

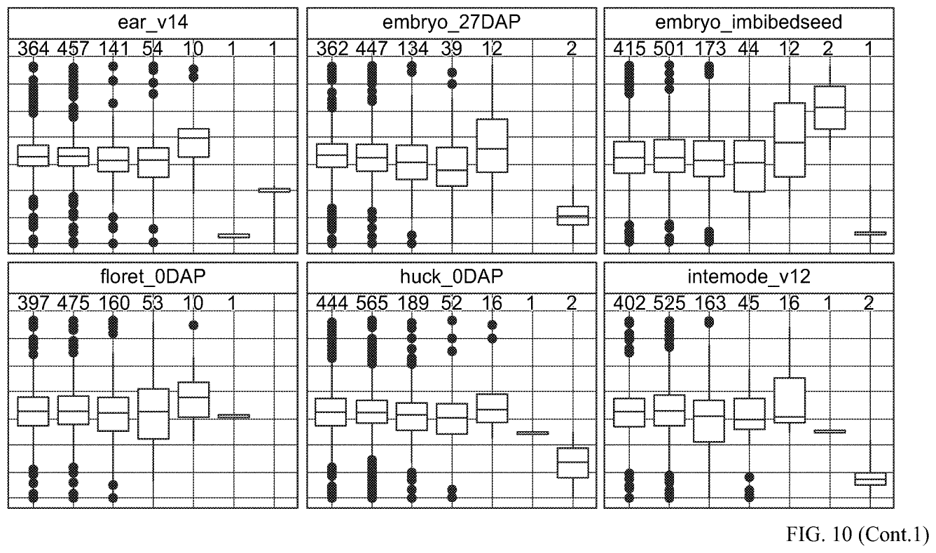

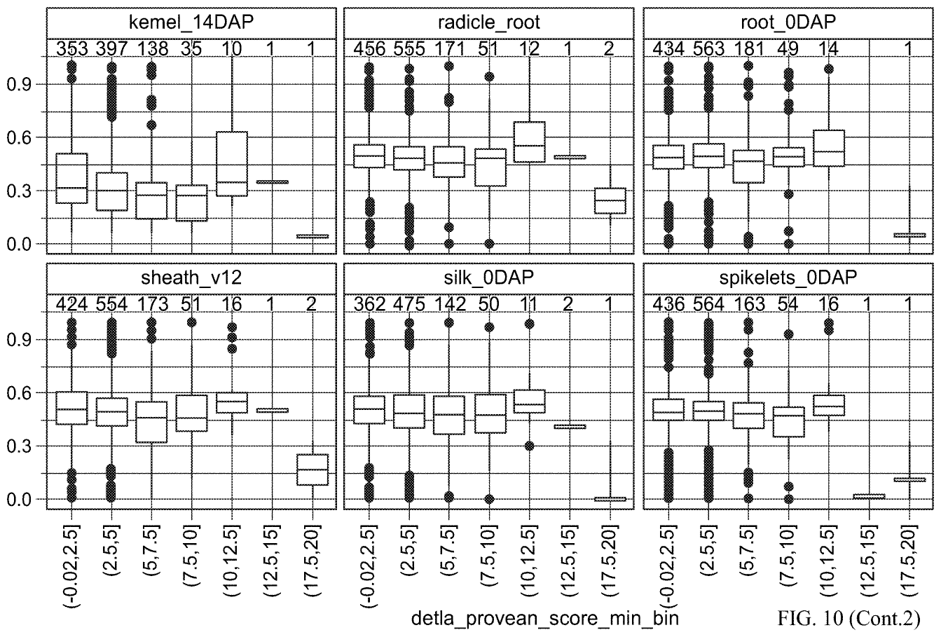

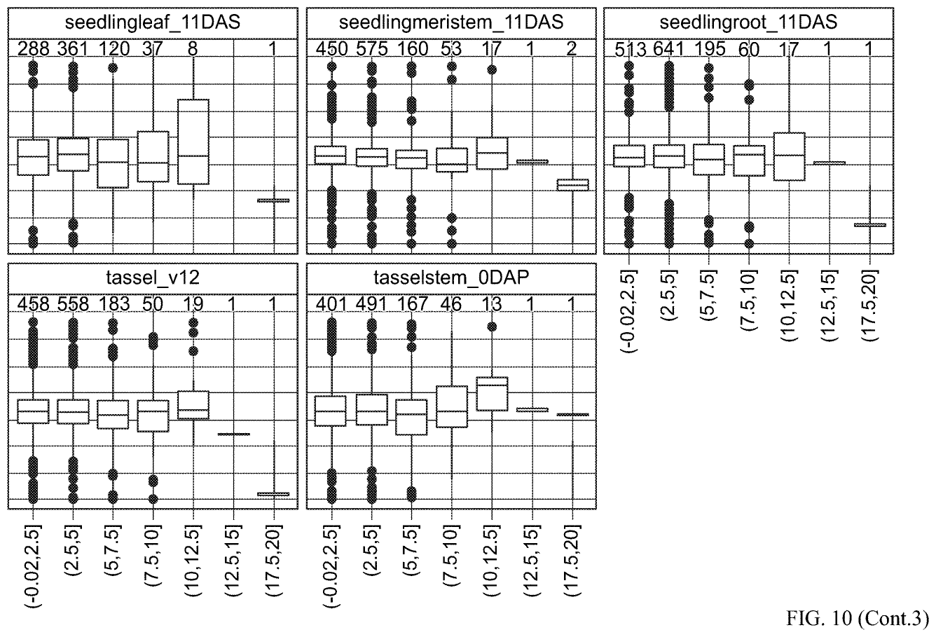

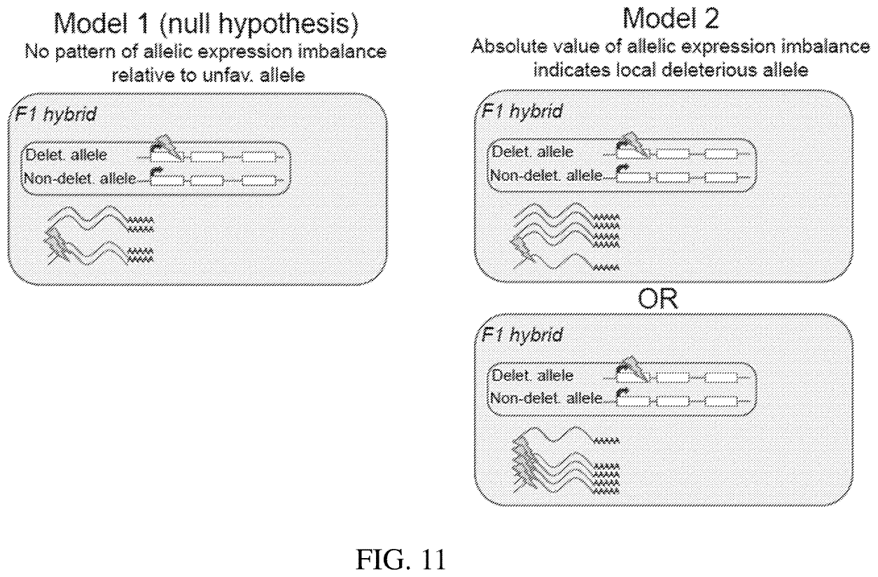

However, as shown in FIG. 10, results also show that deleterious alleles are not necessarily avoided for expression. When there is a big difference between F1's two parental allele fitness levels, the deleterious allele is either avoided for expression or overexpressed, indicating that it is the absolute magnitude of the expression imbalance between the two parental alleles, rather than the direction of the imbalance, that serves as an indicator of possessing a deleterious allele, as illustrated in FIG. 11.

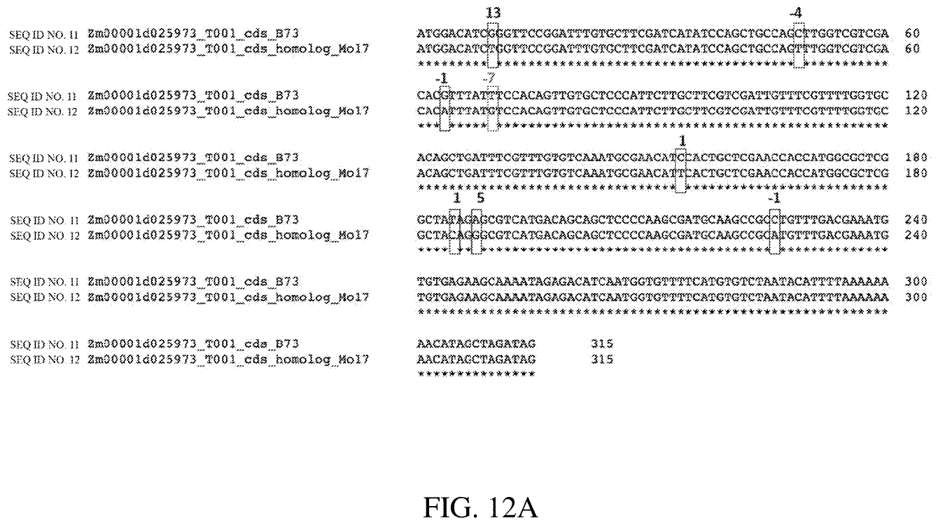

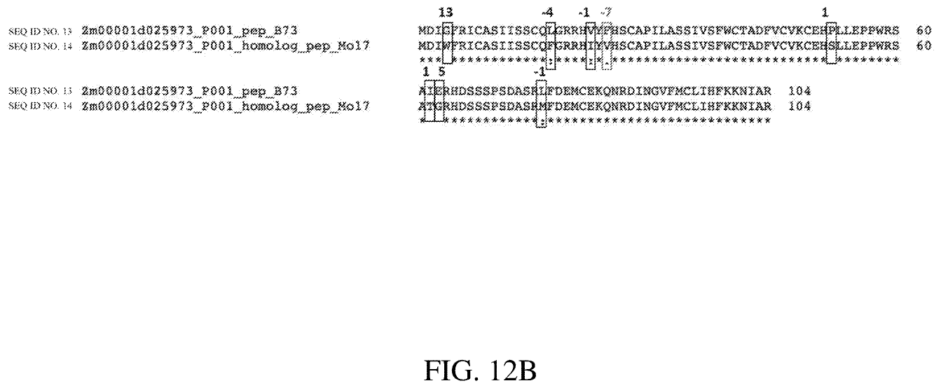

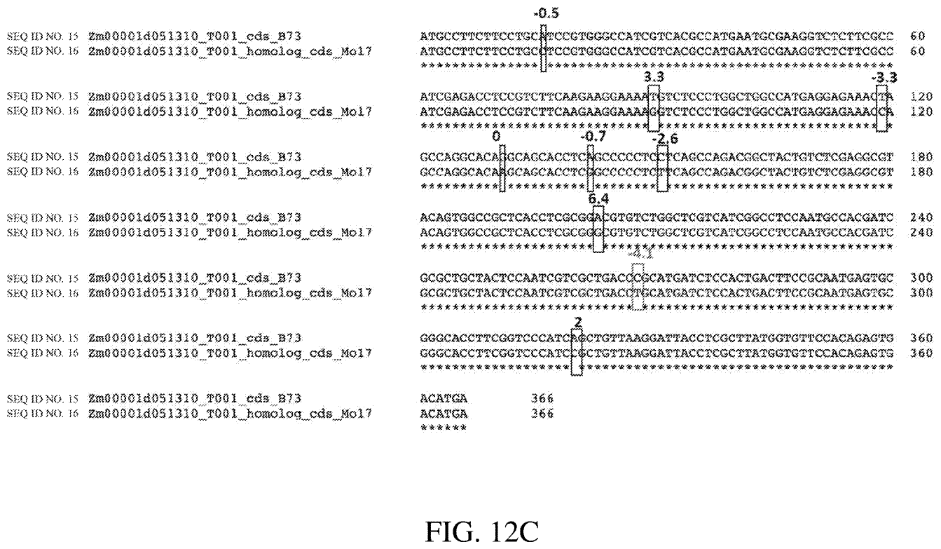

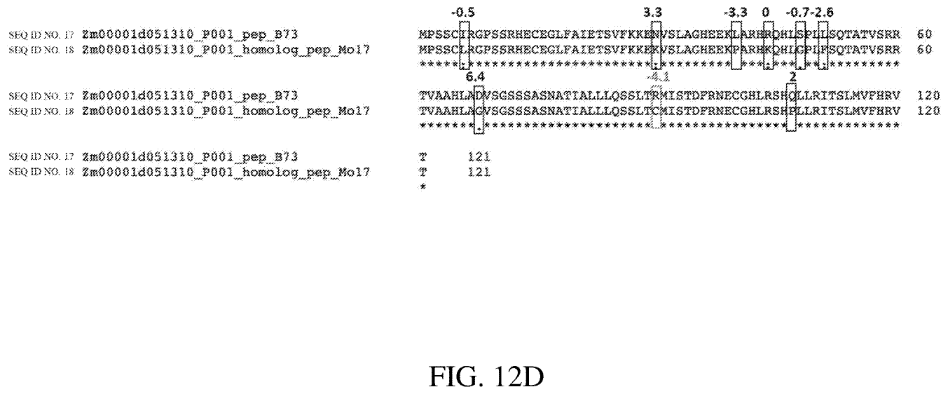

FIGS. 12A-12D show alignment of the CDS and protein sequences (SEQ ID NOs. 11-18) of two genes, Zm00001d025973 and Zm00001d025973, which exhibit strongly biased ASE that are potentially driven by deleterious allele from one copy of the inbred parent. Zm0001d025973 has 9 times expression level of B73 copy of this gene compared to its Mo17 copy, whereas Zm0001d051310 has over 9 times expression level of Mo17 copy of this gene compared to its B73 copy, although both genes contain a more deleterious allele from Mo17 (highlighted in red in FIGS. 12A-12D).

Taken together, this example demonstrates successful implementation of using allele specific expression (ASE) as an endophenotype to identify and prioritize genetic variants in maize. In summary, these findings show that ASE can be used as one endophenotype to help prioritize deleterious sites for modification by genome engineering. Genes exhibiting strong ASE in F1 hybrids are more likely to contain deleterious alleles, and data from many F1 hybrids generated from different inbred parents are useful in prioritizing deleterious sites with observable effect on gene expression levels, plant phenotypes and ultimately fitness. Additionally, these results indicate that when allelic expression bias is observed in hybrid combinations where a specific deleterious allele is heterozygous, this allele should be prioritized for editing in hybrid pairings where the putative deleterious allele is homozygous.

### Example 13: Identification and Assessment of Candidate Deleterious Genetic Variant S277P in Maize Gene Zm00001d047446

This example describes the identification and assessment of candidate deleterious genetic variant S277P in maize (Zea mays) gene Zm00001d047446.

### Materials and Methods

Genetic variants were obtained from the maize haplotype map HapMap 3.2.1 (Bukowski, Robert, et al. “Construction of the third-generation Zea mays haplotype map.” Gigascience 7.4 (2018): gix134).

Predicted variant effects based on evolutionary conservation were obtained from likelihood ratio tests (Knudsen, B. and Miyamoto, M. M., 2001. A likelihood ratio test for evolutionary rate shifts and functional divergence among proteins. Proceedings of the National Academy of Sciences, 98(25), pp. 14512-14517) on multiple sequence alignment (MSA). Specifically, MSA was obtained using plant species beyond maize to calculate conservation at a given locus in maize which contains a genetic variant within maize. Homologous sequences surrounding the given genetic variant locus from non-maize plant species were identified using translated BLAST (tBLASTx) and then aligned by PASTA as implemented in BAD_mutations (Kono et al 2016). Conservation level after accounting for the phylogenetic relationship of the species at the locus was calculated for nucleotide variants segregating in maize with nonsynonymous impact on the resulting protein sequence.

Predicted variant effects based on functional impact of amino acid substitution were obtained using SIFT (Ng, P. C. and Henikoff, S., 2003. SIFT: Predicting amino acid changes that affect protein function. Nucleic acids research, 31(13), pp. 3812-3814) and PROVEAN (Choi, Y., Sims, G. E., Murphy, S., Miller, J. R. and Chan, A. P., 2012. Predicting the functional effect of amino acid substitutions and indels. PloS one, 7(10), p.e46688).

Predicted deleterious genetic variants were then further assessed based on their effect on the changes of gene expression level of gene network partners as an endophenotype. Specifically, the outlier status of mRNA expression (e.g., greater than three standard deviations in a population) in germinating kernel root samples was used as an indicator. See Kremling et al, 2018 Nature, 555(7697), 520-523, and Zhao et al., 2016 The American Journal of Human Genetics, 98(2), 299-309). Expression data for maize line B104 and 290 other sampled individuals was collected and calculated as described by Kremling et al 2018 with network relations as described and calculated using XGboost in Zhou et al 2020.

### Results

A candidate deleterious genetic variant S277P was identified at the position Chr9: 130363251 in Zea mays B73 reference genome AGPv4 and position 277 in the CDS of Zm00001d047446_T002, which is a missense variant leading to a change in genetic sequence from ‘T’ to ‘C’ with an ancestral state of ‘T’ and leading to a change in codon from TCT to CCT and amino acid of serine to proline.

In maize reference line B104, the locus at the position Chr9: 130363251 in Zea mays B73 reference genome AGPv4 and position 277 in the CDS is the derived allele ‘C’. This variant is illustrated below and is flanked by sequence (focal variant shown underlined and bolded below) in sequence from 9:130363150-130363351(+):

(SEQ ID NO. 19)
AAGTCTGTTTGTTTTTTTTAATTTCATAAACTTATTAAAATGTCGCAGG
CCAATTTTGGACCCTATTGCTTCTGTATTCCACAAACTTTTCTGTGGGC
GA [symbol] CTGCAAGACCTGAAGGCACAGGCCAGACATTGGATGGGTCACAGTT
CCCTGGTTCAGGCTCCACTGAGGCAAACAGGAGGAGGTGCGGATTCCCT
TTTTC

The focal gene is (Zm00001d047446_T002), a protein coding gene of unknown function with coordinates of Chr9: 130358116 . . . 130365373 in Zea mays B73 reference genome AGPv4. The variant S277P received a P-value of 0.00072158 using the likelihood ratio test on the multiple sequence alignment. Using SIFT with proteins from UniRef clustered at 90% identity, the variant S277P received a score of 0.03 after being compared to 136 sequences and was classified as deleterious. Using only plant proteins clustered at 90% identity, the variant S277P received a SIFT score of 0.01 which was also classified as deleterious using the SIFT cutoff of 0.05. The S277P variant in Zm00001d047446_T002 also had a PROVEAN score of −1.364 after being compared to 83 other sequences.

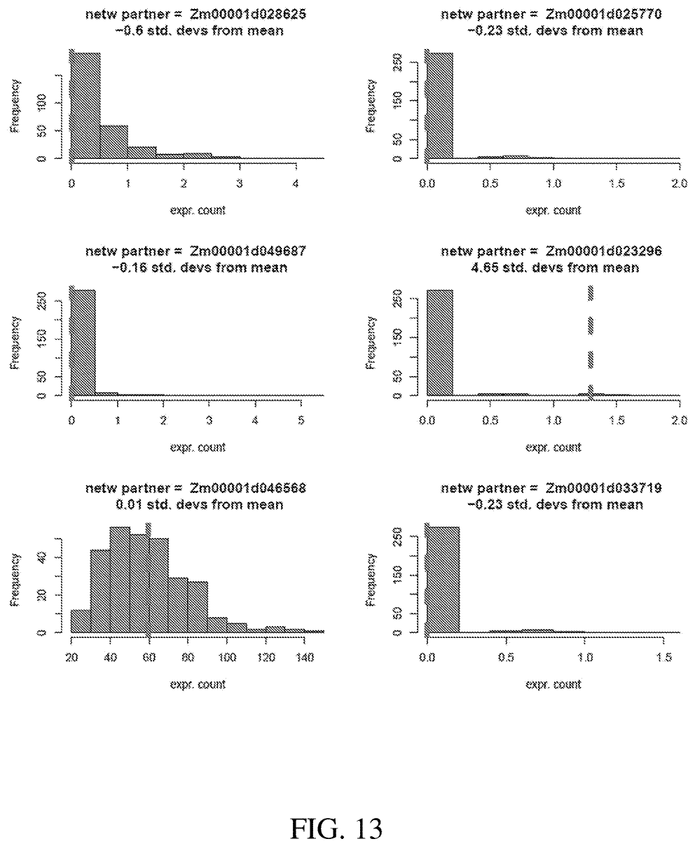

The endophenotypes in the form of the mRNA expression in germinating kernel roots of the corresponding expression network partners of the Zm00001d047446 gene in the maize line B104, which possesses the derived putatively deleterious allele, are displayed as vertical dash lines relative to the population distribution in FIG. 13. For the fourth displayed network partner, Zm00001d023296, the B104 line has expression which is greater than three standard deviations above the population mean, indicating it is an outlier and supporting the interpretation that the aforementioned mutation in Zm00001d047446 is likely deleterious as evidenced by misexpression of the network partner.

This variant is 10 bp away from the PAM recognition site of Cas12a TTTV and as a C->T transition can be corrected using a Cas12a cytosine base editor. Upon editing in a cell-based or plantlet assay to correct this putative deleterious allele described above, the reduction or non reduction of expression outlier status of this expression network partner Zm00001d023296 can be used to corroborate or refute the aforementioned mutation's status as a putatively deleterious allele.

In summary, the predicted candidate deleterious genetic variant exhibited observable effect on gene expression levels of partner genes, leading to its prioritization for further examination of plant phenotypes and ultimately fitness. Taken together, this example demonstrates successful implementation of using the methods of the present disclosure to identify and assess genetic variants in maize.

### Example 14: Identification and Assessment of Candidate Deleterious Genetic Variant P37L in Maize Gene Zm00001d002452

This example describes the identification and assessment of candidate deleterious genetic variant P37L in maize (Zea mays) gene Zm00001d002452.

### Materials and Methods

Genetic variants were obtained from the maize haplotype map HapMap 3.2.1 (Bukowski, Robert, et al. “Construction of the third-generation Zea mays haplotype map.” Gigascience 7.4 (2018): gix134).

Predicted variant effects based on evolutionary conservation were obtained from likelihood ratio tests (Knudsen, B. and Miyamoto, M. M., 2001. A likelihood ratio test for evolutionary rate shifts and functional divergence among proteins. Proceedings of the National Academy of Sciences, 98(25), pp. 14512-14517) on multiple sequence alignment (MSA). Specifically, MSA was obtained using plant species beyond maize to calculate conservation at a given locus in maize which contains a genetic variant within maize. Homologous sequences surrounding the given genetic variant locus from non-maize plant species were identified using translated BLAST (tBLASTx) and then aligned by PASTA as implemented in BAD_mutations (Kono et al 2016). Conservation level after accounting for the phylogenetic relationship of the species at the locus was calculated for nucleotide variants segregating in maize with nonsynonymous impact on the resulting protein sequence.

Predicted variant effects based on functional impact of amino acid substitution were obtained using SIFT (Ng, P. C. and Henikoff, S., 2003. SIFT: Predicting amino acid changes that affect protein function. Nucleic acids research, 31(13), pp. 3812-3814) and PROVEAN (Choi, Y., Sims, G. E., Murphy, S., Miller, J. R. and Chan, A. P., 2012. Predicting the functional effect of amino acid substitutions and indels. PloS one, 7(10), p.e46688).

Predicted deleterious genetic variants were then further assessed based on their effect on the changes of gene expression level of gene network partners as an endophenotype. Specifically, the outlier status of mRNA expression (e.g., greater than three standard deviations in a population) in germinating kernel root samples was used as an indicator. See Kremling et al, 2018 Nature, 555(7697), 520-523, and Zhao et al., 2016 The American Journal of Human Genetics, 98(2), 299-309). Expression data for maize line B104 and 290 other sampled individuals was collected and calculated as described by Kremling et al 2018 with network relations as described and calculated using XGboost in Zhou et al 2020.

### Results

A candidate deleterious genetic variant P37L was identified at the position Chr2: 13071694 in Zea mays B73 reference genome AGPv4 and position 37 in the CDS of Zm00001d002452_T001, which is a missense variant leading to a change in genetic sequence from ‘G’ to ‘A’ with an ancestral state of ‘G’ and leading to a change in codon from CCG to CTG and amino acid of proline to lysine (note gene is on negative strand G/A SNP leads to CCG/CTG codon change).

In maize reference line B104, the locus at the position Chr2: 13071694 in Zea mays B73 reference genome AGPv4 and position 37 in the CDS is the derived allele ‘T’. This variant is illustrated below and is flanked by sequence (focal variant shown underlined and bolded below) in sequence from 2:13071593-13071794(−):

(SEQ ID NO. 20)
GGCGCAGCCTACTTCCGATGCTGTCGTCGACGAGGGAAGCGGCGGGAAG
AGCATCGTCGCGTCCCCCTGGAGCTGCCACTCGTCCGCGGCGGCCGTGG
AC [symbol] GCGTGTCCGCGGCGTTTCCAGGGATGGCTCCGCCGGACCGGACGAT
GTCCGTGAGGTCTCCGCCGCCGGCCTGGTCGCCCTCCATCCTCGGAAGG
AAGTA

The focal gene is (Zm00001d002452), a protein coding gene encoding the WRKY transcription factor wrky70 with coordinates of Chr2: 13066792 . . . 13073303 in Zea mays B73 reference genome AGPv4. The variant P37L received a P value of 0.0.001231309 using the likelihood ratio test on the multiple sequence alignment above. Using SIFT with proteins from UniRef clustered at 90% identity, the variant P37L received a score of 0.03 after being compared to 33 sequences and is classified as deleterious. Using only plant proteins clustered at 90% identity, the variant P37L received a SIFT score of 0.07. The P37L variant in Zm00001d002452_T001 also had a PROVEAN score of −2.446 after being compared to 67 other sequences.

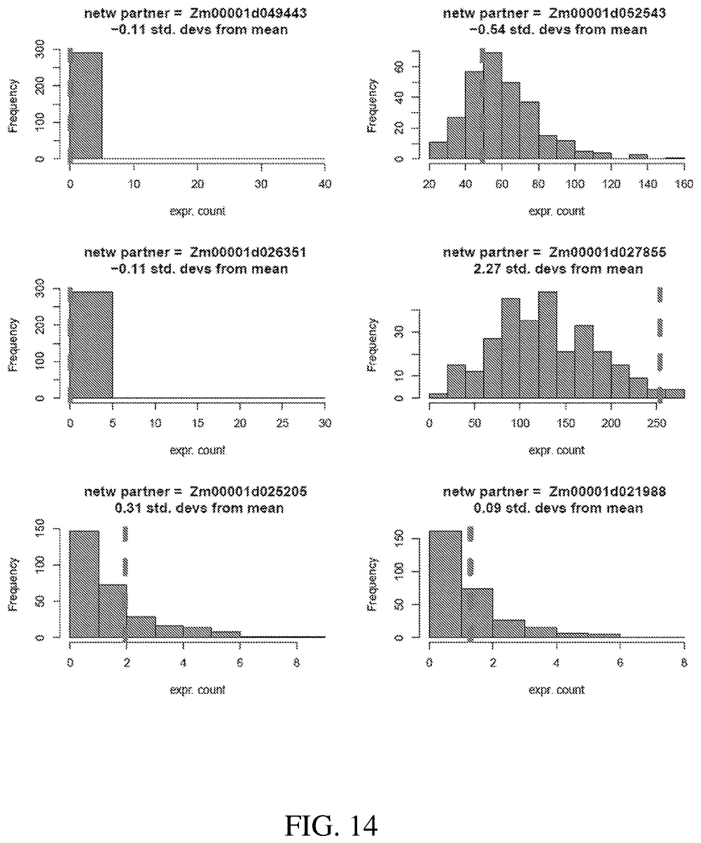

The endophenotypes in the form of mRNA expression in germinating kernel roots of the first six corresponding expression network partners of the Zm00001d002452 gene in the maize line B104, which possesses the derived putatively deleterious allele, are displayed as vertical dash lines relative to the population distribution in the FIG. 14. However, the line of interest, B104, does not display expression outlier status defined by being greater than 3 standard deviations from the population mean for any of the first six expression network partners, which leads to its deprioritization as a deleterious allele although it is within the targetable window of a base editor as described below.

This variant is 15 bp away from the PAM recognition site of Cas12a TTTV and as a G->A transition can be corrected using a Cas12a adenine base editor.

In summary, a candidate deleterious genetic variant was identified. However, based on the magnitude of the effect, this candidate variant did not exhibit significant effect on gene expression levels of partner genes, which leads to its deprioritization (i.e., an unlikely candidate) for further downstream examination of plant phenotypes. Taken together, this example demonstrates successful implementation of using the methods of the present disclosure to identify and assess genetic variants in maize.

### Example 15: Identification and Assessment of Candidate Deleterious Genetic Variant V232I in Maize Gene Zm00001d016008

This example describes the identification and assessment of candidate deleterious genetic variant V232I in maize (Zea mays) gene Zm00001d016008.

### Materials and Methods

Genetic variants were obtained from the maize haplotype map HapMap 3.2.1 (Bukowski, Robert, et al. “Construction of the third-generation Zea mays haplotype map.” Gigascience 7.4 (2018): gix134).

Predicted variant effects based on evolutionary conservation were obtained from likelihood ratio tests (Knudsen, B. and Miyamoto, M. M., 2001. A likelihood ratio test for evolutionary rate shifts and functional divergence among proteins. Proceedings of the National Academy of Sciences, 98(25), pp. 14512-14517) on multiple sequence alignment (MSA). Specifically, MSA was obtained using plant species beyond maize to calculate conservation at a given locus in maize which contains a genetic variant within maize. Homologous sequences surrounding the given genetic variant locus from non-maize plant species were identified using translated BLAST (tBLASTx) and then aligned by PASTA as implemented in BAD_mutations (Kono et al 2016). Conservation level after accounting for the phylogenetic relationship of the species at the locus was calculated for nucleotide variants segregating in maize with nonsynonymous impact on the resulting protein sequence.

Predicted variant effects based on functional impact of amino acid substitution were obtained using SIFT (Ng, P. C. and Henikoff, S., 2003. SIFT: Predicting amino acid changes that affect protein function. Nucleic acids research, 31(13), pp. 3812-3814) and PROVEAN (Choi, Y., Sims, G. E., Murphy, S., Miller, J. R. and Chan, A. P., 2012. Predicting the functional effect of amino acid substitutions and indels. PloS one, 7(10), p.e46688).

Predicted deleterious genetic variants were then further assessed based on their effect on the changes of gene expression level of gene network partners as an endophenotype. Specifically, the outlier status of mRNA expression (e.g., greater than three standard deviations in a population) in germinating kernel root samples was used as an indicator. See Kremling et al, 2018 Nature, 555(7697), 520-523, and Zhao et al., 2016 The American Journal of Human Genetics, 98(2), 299-309). Expression data for maize line B104 and 290 other sampled individuals was collected and calculated as described by Kremling et al 2018 with network relations as described and calculated using XGboost in Zhou et al 2020.

### Results

A candidate deleterious genetic variant V232I was identified at the position 5:139152841 in Zea mays B73 reference genome AGPv4 and position 232 in the CDS of Zm00001d016008_T001, which is a missense variant leading to a change in genetic sequence from ‘G’ to ‘A’ with an ancestral state of ‘G’ and leading to a change in codon from GTC to ATC and amino acid of valine to isoleucine.

In reference line B104, the locus at the position Chr5:139152841 in Zea mays B73 reference genome AGPv4 and position 232 in the CDS is the derived allele ‘A’. This variant is illustrated below and is flanked by sequence (focal variant shown underlined and bolded below) in sequence from 5:139152740-139152941(+):

(SEQ ID NO. 21)
AACAATCTACTTGCAGTGCAATATTTCTAAATTGTACTTGCAGTGCAAG
ATTTCAAATCAATCTACTTACCGTGCCAGATTTCAGGGGCTTAGACAGG
AT [symbol] TCATTGCAGACCTTGTGGATCAATGCCGTTCTTACAAGCAAAGAGT
AGTGCAGCTTGTCAACAGTACCTCGTAAGTTACCTTGATGACTCTTTTC
TAGTT

The focal gene is (Zm00001d016008), a protein coding gene of unknown function with coordinates of Chr5: 139146155 . . . 139157520 in Zea mays B73 reference genome AGPv4. The variant V232I received a P-value of 0.002413646597 using the likelihood ratio test on the multiple sequence alignment above. Using SIFT with proteins from UniRef clustered at 90% identity, the variant V232I received a score of 0.01 after being compared to 172 sequences and is classified as deleterious. Using only plant proteins clustered at 90% identity, the variant V232I received a SIFT score of 0. The V232I variant in Zm00001d016008 also had a PROVEAN score of −0.3 after being compared to 64 other sequences.

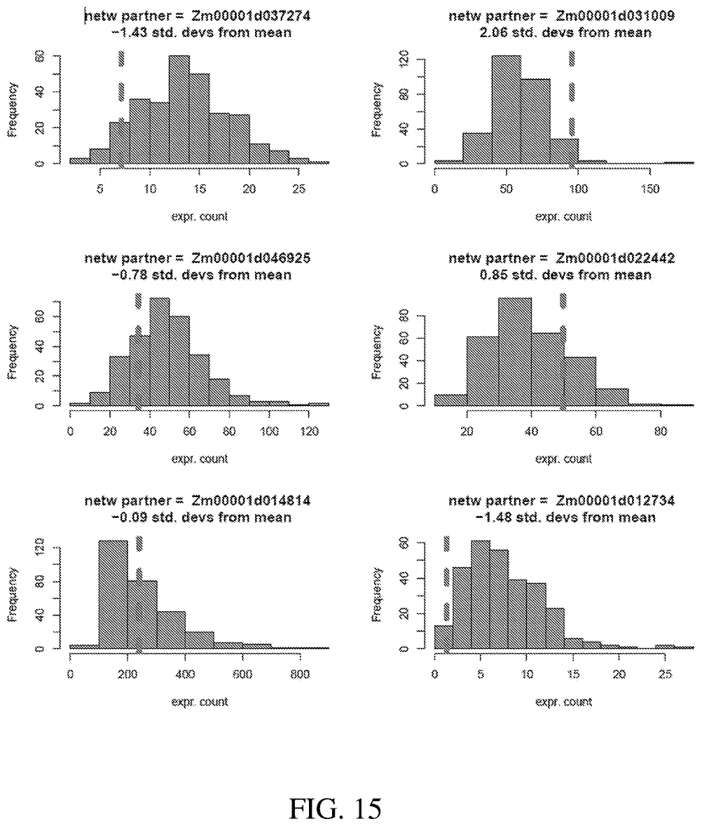

The endophenotypes in the form of mRNA expression in germinating kernel roots of the first six corresponding expression network partners of the Zm00001d016008 gene in the maize line B104, which possesses the derived putatively deleterious allele, are displayed as vertical dash lines relative to the population distribution in FIG. 15. However, the line of interest, B104, does not display expression outlier status defined by being greater than three standard deviations from the population mean for any of the first six expression network partners, which leads to its deprioritization as a deleterious allele although it is within the targetable window of a base editor as described below.

This variant is 18 bp away from the PAM recognition site of Cas12a TTTV and as a G->A transition can be corrected using a Cas12a adenine base editor.

In summary, a candidate deleterious genetic variant was identified. However, based on the magnitude of the effect, this candidate variant did not exhibit significant effect on gene expression levels of partner genes, which leads to its deprioritization (i.e., an unlikely candidate) for further downstream examination of plant phenotypes. Taken together, this example demonstrates successful implementation of using the methods of the present disclosure to identify and assess genetic variants in maize.

What is claimed is:

1. A method for improving performance of an organism, comprising:
a) providing a plurality of genetic variants in the genome of the organism;
b) predicting the effects of the genetic variants on the performance of the organism using a statistical model;
c) altering one or more of the genetic variants in the genome of the organism;
d) identifying an impact of the alteration on an endophenotype, wherein the endophenotype is a quantifiable phenotype at the sub-organismal level that can be measured by a biochemical, gene expression, or protein level assay, or visually via microscopy;
e) updating the statistical model using the identified endophenotypic impact;
f) optionally repeating steps c) to e) for one or more times;
g) determining the genetic variants having a predicted negative effect on the performance of the organism using the updated statistical model; and
h) modifying in the genome one or more of the genetic variants having a predicted negative effect on the performance of the organism, thereby improving performance of an organism.

2. A method for selecting an organism with improved performance in a population, comprising:
a) providing a population of organisms;
b) providing a plurality of genetic variants of the population;
c) predicting the effects of the genetic variants on the performance of the organisms using a statistical model;
d) altering one or more of the genetic variants in one or more of the organisms;
e) identifying an impact of the alteration on an endophenotype, wherein the endophenotype is a quantifiable phenotype at the sub-organismal level that can be measured by a biochemical, gene expression, or protein level assay, or visually via microscopy;
f) updating the statistical model using the identified endophenotypic impact;
g) optionally repeating steps d) to f) for one or more times;
h) determining the genetic variants having predicted positive effects on the performance of the organisms using the updated statistical model; and
i) selecting in the population an organism comprising one or more of the genetic variants having predicted positive effects on the performance of the organisms, selecting an organism with improved performance in a population.

3. A method for removing an underperforming organism from a population, comprising:
a) providing a population of organisms;
b) providing a plurality of genetic variants of the population;
c) predicting the effects of the genetic variants on the performance of the organisms using a statistical model;
d) altering one or more of the genetic variants in one or more of the organisms;
e) identifying an impact of the alteration on an endophenotype, wherein the endophenotype is a quantifiable phenotype at the sub-organismal level that can be measured by a biochemical, gene expression, or protein level assay, or visually via microscopy;
f) updating the statistical model using the identified endophenotypic impact;
g) optionally repeating steps d) to f) for one or more times;
h) determining the genetic variants having predicted negative effects using the updated statistical model; and
i) removing from the population an organism comprising one or more of the genetic variants having predicted negative effects on the performance of the organisms, thereby removing an underperforming organism from a population.

4. A method for prioritizing genetic variants based on predicted effects on performance of an organism, comprising:
a) providing a plurality of genetic variants in the genome of the organism;
b) predicting the effects of the genetic variants on the performance of the organism using a statistical model;
c) altering one or more of the genetic variants in the genome of the organism;
d) identifying an impact of the alteration on an endophenotype, wherein the endophenotype is a quantifiable phenotype at the sub-organismal level that can be measured by a biochemical, gene expression, or protein level assay, or visually via microscopy;
e) updating the statistical model using the identified endophenotypic impact;
f) optionally repeating steps c) to e) for one or more times; and
g) prioritizing the genetic variants based on the magnitudes of the predicted effects on the performance of organism using the updated statistical model.

5. The method of any one of claims 1-4, wherein the organism is maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, or a biomass crop.

6. The method of claim 5, wherein the performance of the organism is yield, overall fitness, biomass, photosynthetic efficiency, nutrient use efficiency, heat tolerance, drought tolerance, herbicide tolerance, or disease resistance.

7. The method of any one of claims 1-4, wherein the organism is cattle, sheep, goat, horse, pig, chicken, duck, goose, rabbit, or fish.

8. The method of claim 7, wherein the performance of the organism is growth rate, feed use efficiency, meat yield, meat quality, milk yield, milk quality, egg yield, egg quality, wool yield, or wool quality.

9. The method of any one of claims 1-8, wherein the performance is a quantitative trait.

10. The method of any one of claims 1-9, wherein the genetic variants are identified by a linkage study.

11. The method of any one of claims 1-9, wherein the genetic variants are identified by an association study.

12. The method of claim 11, wherein the association study is a genome-wide association study (GWAS) or a transcriptome-wide association study (TWAS).

13. The method of any one of claims 1-12, wherein the statistical model is a linear regression model, a logistic regression model, a ridge regression model, a lasso regression model, an elastic net regression model, a decision tree model, a gradient boosted tree model, a neural network model, or a support vector machine (SVM) model.

14. The method of any one of claims 1-13, wherein the statistical model comprises a feature based on evolutionary conservation of the genetic variants.

15. The method of claim 14, wherein the evolutionary conservation is determined by sequence alignment in a genic or an intergenic region.

16. The method of any one of claims 1-13, wherein the statistical model comprises a feature based on functional impact of amino acid change of the genetic variants.

17. The method of claim 16, wherein the functional impact of amino acid change is weighted according to the blocks substitution matrix (BLOSUM).

18. The method of any one of claims 1-13, wherein the statistical model comprises a feature based on functional impact of protein conformation and/or stability of the genetic variants.

19. The method of claim 18, wherein the functional impact of protein conformation and/or stability is determined by a Monte Carlo search for minimal free energy.

20. The method of claim 18, wherein the functional impact of protein conformation and/or stability is predicted by learning a representation of amino acid order from existing proteins in higher dimensional space.

21. The method of any one of claims 1-13, wherein the statistical model comprises a feature based on adjacency to a selective sweep region of the genetic variants.

22. The method of claim 21, wherein the selective sweep region is determined by a decrease of pairwise nucleotide diversity it or linkage disequilibrium relative to the rest of the genome.

23. The method of any one of claims 1-13, wherein the statistical model comprises a feature based on outlier status of an endophenotype associated with a genetic variant that is physically proximal or proximal within a gene network.

24. The method of any one of claims 1-23, wherein the alteration is achieved by genome editing.

25. The method of claim 24, wherein the genome editing is achieved by a clustered regularly interspersed short palindromic repeats (CRISPR) system, a transcription activator-like effector nuclease (TALEN) system, or a zinc finger nuclease (ZFN) system.

26. The method of any one of claims 1-23, wherein the alteration is achieved by creation of novel haplotype combinations from genetic recombination during meiosis.

27. The method of any one of claims 1-26, wherein the endophenotype is messenger RNA (mRNA) abundance.

28. The method of any one of claims 1-26, wherein the endophenotype is gene transcript splicing ratio.

29. The method of any one of claims 1-26, wherein the endophenotype is protein abundance.

30. The method of any one of claims 1-26, wherein the endophenotype is micro RNA (miRNA) or small RNA (siRNA) abundance.

31. The method of any one of claims 1-26, wherein the endophenotype is translational efficiency.

32. The method of any one of claims 1-26, wherein the endophenotype is ribosome occupancy.

33. The method of any one of claims 1-26, wherein the endophenotype is protein modification.

34. The method of any one of claims 1-26, wherein the endophenotype is metabolite abundance.

35. The method of any one of claims 1-26, wherein the endophenotype is allele specific expression (ASE).

36. An organism with improved performance produced or selected by the method of any one of claims 1-35.

37. A computer-implemented method for assessing genetic variants for use in genetic improvement of an organism, comprising:
a) receiving a dataset comprising a plurality of genetic variants of the organism; and
b) performing a prediction of the effects of the genetic variants using a statistical model comprising one or more initial rules that associate the genetic variants with performance of the organism.

38. The method of claim 37, further comprising updating the statistical model with one or more new rules, wherein the one or more new rules are based on data generated from an endophenotype, wherein the endophenotype is a quantifiable phenotype at the sub-organismal level that can be measured by a biochemical, gene expression, or protein level assay, or visually via microscopy.

39. The method of any one of claims 37-38, wherein the statistical model is a linear regression model, a logistic regression model, a ridge regression model, a lasso regression model, an elastic net regression model, a decision tree model, a gradient boosted tree model, a neural network model, or a support vector machine (SVM) model.

40. The method of any one of claims 37-39, wherein the one or more initial rules or the one or more new rules comprise evolutionary conservation, functional impact of amino acid change, functional impact of protein conformation and/or stability, adjacency to selective sweep regions, outlier status of an endophenotype associated with a genetic variant that is physically proximal or proximal within a gene network, or a combination thereof.

41. The method of any one of claims 38-40, wherein the endophenotype is messenger RNA (mRNA) abundance, gene transcript splicing ratio, protein abundance, micro RNA (miRNA) or small RNA (siRNA) abundance, translational efficiency, ribosome occupancy, protein modification, metabolite abundance, allele specific expression (ASE), or a combination thereof.

42. A computer-readable storage medium storing computer-executable instructions, comprising:
a) instructions for applying a statistical model to a dataset,
wherein the dataset comprises a plurality of genetic variants of an organism, and
wherein the statistical model comprises one or more initial rules that associate the genetic variants with performance of the organism; and
b) instructions for calculating an effect value related to the performance of the organism for each of the genetic variants.

43. The computer-readable storage medium of claim 42, further comprising instructions for updating the statistical model with at least one new rule, wherein at least one new rule is based on data generated from an endophenotype, wherein the endophenotype is a quantifiable phenotype at the sub-organismal level that can be measured by a biochemical, gene expression, or protein level assay, or visually via microscopy.

44. The computer-readable storage medium of any one of claims 42-43, wherein the statistical model is a linear regression model, a logistic regression model, a ridge regression model, a lasso regression model, an elastic net regression model, a decision tree model, a gradient boosted tree model, a neural network model, or a support vector machine (SVM) model.

45. The computer-readable storage medium of any one of claims 42-44, wherein the one or more initial rules or the one or more new rules comprise evolutionary conservation, functional impact of amino acid change, functional impact of protein conformation and/or stability, adjacency to selective sweep regions, outlier status of an endophenotype associated with a genetic variant that is physically proximal or proximal within a gene network, or a combination thereof.

46. The computer-readable storage medium of any one of claims 43-45, wherein the endophenotype is messenger RNA (mRNA) abundance, gene transcript splicing ratio, protein abundance, micro RNA (miRNA) or small RNA (siRNA) abundance, translational efficiency, ribosome occupancy, protein modification, metabolite abundance, allele specific expression (ASE), or a combination thereof.

47. A system for assessing genetic variants for use in genetic improvement of an organism, comprising:
a) a computer-readable storage medium storing a database comprising a plurality of genetic variants of the organism;
b) a computer-readable storage medium storing computer-executable instructions, comprising:
i) instructions for applying a statistical model to the dataset,
wherein the statistical model comprises one or more initial rules that associate the genetic variants with performance of the organism; and
ii) instructions for calculating an effect value related to the performance of the organism for each of the genetic variants; and
c) a processor configured to execute the computer-executable instructions stored in the computer-readable storage medium.

48. The system of claim 47, wherein the computer-readable storage medium further comprises instructions for updating the statistical model with one or more new rules, wherein the one or more new rules are based on data generated from an endophenotype, wherein the endophenotype is a quantifiable phenotype at the sub-organismal level that can be measured by a biochemical, gene expression, or protein level assay, or visually via microscopy.

49. The system of any one of claims 47-48, wherein the statistical model is a linear regression model, a logistic regression model, a ridge regression model, a lasso regression model, an elastic net regression model, a decision tree model, a gradient boosted tree model, a neural network model, or a support vector machine (SVM) model.

50. The system of any one of claims 47-49, wherein the one or more initial rules or the one or more new rules comprise evolutionary conservation, functional impact of amino acid change, functional impact of protein conformation and/or stability, adjacency to selective sweep regions, outlier status of an endophenotype associated with a genetic variant that is physically proximal or proximal within a gene network, or a combination thereof.

51. The system of any one of claims 48-50, wherein the endophenotype is messenger RNA (mRNA) abundance, gene transcript splicing ratio, protein abundance, micro RNA (miRNA) or small RNA (siRNA) abundance, translational efficiency, ribosome occupancy, protein modification, metabolite abundance, allele specific expression (ASE), or a combination thereof.

52. A method for prioritizing genetic variants, comprising:
a) providing a plurality of genetic variants in the genome of an organism;
b) predicting the effects of the genetic variants on the performance of the organism using an endophenotype; and
c) prioritizing the genetic variants based on the magnitudes of the predicted effects on the performance of the organism.

53. The method of claim 52, further comprising altering one or more of the prioritized genetic variants in the organism.

54. The method of claim 52, further comprising selecting one or more of the prioritized genetic variants from a population of the organisms.

55. The method of any one of claims 52-54, wherein the endophenotype is allele specific expression (ASE).

56. The method of any one of claims 52-55, wherein the statistical model comprises calculating the effect of a genetic variant on the biological function of a protein.

57. The method of claim 56, wherein the calculated effect of a genetic variant is a likelihood ratio test P-value, a Protein Variation Effect Analyzer (PROVEAN) score, or a Sorting Intolerant from Tolerant (SIFT) score.

58. The method of any one of claims 52-57, wherein the organism is maize, wheat, barley, oat, rice, soybean, oil palm, safflower, sesame, tobacco, flax, cotton, sunflower, pearl millet, foxtail millet, sorghum, canola, cannabis, a vegetable crop, a forage crop, an industrial crop, a woody crop, or a biomass crop.

59. The method of claim 52-58, wherein the organism is hybrid maize.

60. The method of any one of claims 52-59, wherein the performance of the organism is yield, overall fitness, biomass, photosynthetic efficiency, nutrient use efficiency, heat tolerance, drought tolerance, herbicide tolerance, or disease resistance.

61. The method of any one of claims 52-60, wherein the genetic variants comprise a deleterious allele that confers or correlates with a negative effect to the performance of the organism.

62. The method of claim 61, wherein the deleterious allele is overexpressed or underexpressed in the organism in comparison to a control organism.

63. The method of claim 62, wherein the control organism is an inbred line.

64. The method of any one of claims 52-63, wherein the genetic variants are homozygous or heterozygous in the organism.

65. The method of any one of claims 52-64, wherein the genetic variants comprise a deleterious allele that is homozygous in the organism.

66. The method of any one of claims 52-65, wherein the prioritized genetic variants comprise a target for gene editing.

67. The method of any one of claims 52-66, wherein the prioritized genetic variants comprise a deleterious allele homozygous in the organism that is used as a target for gene editing.

68. The method of any one of claims 66-67, wherein the gene editing is achieved by a zinc finger nuclease (ZFN) system, a transcription activator-like effector nuclease (TALEN) system, or a clustered regularly interspersed short palindromic repeats (CRISPR) system.
